(19) United States
(12) Patent Application Publication
(10) Pub. No.: US 2013/0116343 A1
(43) Pub. Date: May 9, 2013

(54) Salivary Protein Markers for Detection of Breast Cancer

(71) Applicants: Charles F. Streckfus, Seabrook, TX (US); William P. Dubinsky, Houston, TX (US); Lenora Bigler, Houston, TX (US); Karen A. Storthz, Houston, TX (US); Daniel Arreola, Houston, TX (US)

(72) Inventors: Charles F. Streckfus, Seabrook, TX (US); William P. Dubinsky, Houston, TX (US); Lenora Bigler, Houston, TX (US); Karen A. Storthz, Houston, TX (US); Daniel Arreola, Houston, TX (US)

(73) Assignee: BOARD OF REGENTS OF THE UNIVERSITY OF TEXAS SYSTEM, Austin, TX (US)

(21) Appl. No.: 13/642,587

(22) PCT Filed: Apr. 21, 2011
(86) PCT No.: PCT/US11/33426

§ 371 Date: Jan. 2, 2013

Related U.S. Application Data
(60) Provisional application No. 61/326,413, filed on Apr. 21, 2010.

(51) Int. Cl. G01N 33/68

(52) U.S. Cl. CPC G01N 33/6893
USPC 514/789; 436/501; 435/7.92; 435/7.4

(57) ABSTRACT

A method of diagnosing a patient's risk of breast cancer comprises measuring in a saliva sample from the patient a concentration of at least a first protein marker, wherein the first protein marker is either ubiquitin or cytochrome p450, to provide a set of test data comprising a concentration value of each protein marker in the saliva sample. The test values are then compared to a reference panel comprising a Reference Control Value and a Reference Breast Cancer Value, and a diagnosis of either increased or decreased risk of breast cancer is determined for the patient based on a result of that comparison. A test kit for identifying a person at increased risk of breast cancer is also provided.

### BACKGROUND

1. Technical Field

The invention generally relates to methods and compositions for diagnosing breast cancer, and, more particularly, to such methods and compositions which use the differential expression of protein markers (biomarkers) in the saliva of an individual to assess risk of breast cancer, and in some cases differentiate among ductal carcinoma in situ of the breast, benign fibroadenoma and non-cancerous breast tissue in an individual.

2. Description of Related Art

Conventional physical examination and mammography are useful screening procedures for the early detection of breast cancer. However, they can produce a substantial percentage of false positive and false negative results, especially in women with dense parenchymal breast tissue. Consequently, current screening procedures can result in a high percentage of false positive results which are then followed by both physically and emotionally traumatic but negative biopsy results. There is also a demonstrated lack of sensitivity in detecting cancerous lesions in younger women, yielding a significant percentage of false negatives. Although advanced technology in the field of mammography allows more reliable detection of small lesions of the breast, a clear need exists for added modalities of screening, particularly for diagnosing cancer in younger women.

There has been extensive use of immunohistochemistry to detect expression of specific biomarkers as a potential adjunct diagnostic procedure for certain tumors. Primarily, the markers have been found in serum and in tissues. Protein tumor markers such as c-erbB-2 (erb) and Cathespin-D (CD) have been assayed in tissue and shown to correlate with aggressive lesions.

The term “proteomics” was originally defined to represent the analysis of the entire protein component of a cell or tissue, but that term now encompasses the study of expressed proteins, including identification and elucidation of the structure-function relationship under healthy conditions and disease conditions. In combination with genomics, proteomics can provide a holistic understanding of the biology underlying disease processes. Protein expression and function are subject to modulation through transcription as well as through posttranscriptional and translational events. Multiple RNA species can result from one gene through a process of differential splicing. Additionally, there are more than 200 post-translation modifications that proteins could undergo that affect function, protein-protein and nuclide-protein interaction, stability, targeting half-life, and so forth, all contributing to a potentially large number of protein products from one gene.

Technological advancements have benefited proteomic research to the point where saliva is now being assayed for protein content using the latest available proteomic technology (Wong, D. T. JADA. 2006, 137, 313-21). Using immunological techniques, it has been demonstrated that saliva from breast cancer patients exhibited elevated levels of c-erbB-2, CA 15-3, EGFR, cathepsin D and p53, suggesting that there is communication between the breast tumor and the salivary gland. In single analyte reports, additional low-abundance proteins such as HER2/neu, Waf-1, pantropic p53, EGFR and cathepsin D were found to be altered. Recently, the tumor biomarkers CA 125, c-erB-2 (erb) and Cathespin-D (CD) have been detected in saliva and employed in a diagnostic panel for the initial detection and follow-up screening of breast cancer patients (Streckfus, et al., Clin Cancer Res. 2000, 6(6), 2363-70; Streckfus, et al. Cancer Invest. 2000, 18(2), 101-9).

Methods of analysis that have been applied to salivary proteins include surface-enhanced laser desorption and ionization time-of-flight (SELDI-TOF) protein chips combines matrix-assisted laser desorption and ionization TOF mass spectrometry (MS) to surface chromatography. This technology uses sample chips that display various kinds of chemically enriched and active surfaces that bind protein molecules based on established principles, such as ion exchange chromatography, metal ion affinity, and hydrophobic affinity. The technique enables rapid and high-throughput detection of critical proteins and peptides directly from crude mixtures without labor-intensive preprocessing. Furthermore, SELDI-TOF-MS is sensitive and requires only small amounts of sample compared with other proteomic techniques because surface charge is the result of weak acidic and basic amino acids within the protein, binding of the protein to the array is highly pH dependent (Schipper, et al. J Chromatogr B Analyt Technol Biomed Life Sci 2007; 847: 46-53). This method has been used to identify the presence of cancer biomarkers in saliva (Streckfus, et al. J Oral Pathol Med 2006; 35:292-300; Streckfus and Dubinsky, Expert Rev Proteomics 2007; 4(3):329-32; Streckfus, et al. Cancer Invest 2008; 26(2):159-67; Streckfus, et al. Clin Cancer Res 2000; 6(6):2363-70). There is continuing interest in the development of adjunct diagnostic procedures to enhance breast cancer screening.

### BRIEF SUMMARY

In accordance with certain embodiments of the invention, a method of diagnosing a patient's risk of breast cancer comprises measuring in a saliva sample from the patient a concentration of at least a first protein marker (e.g., via an immunoassay), wherein the first protein marker is either ubiquitin or cytochrome p450, to provide a set of test data comprising a concentration value of each protein marker in the saliva sample; comparing the test data to a reference panel comprising a Reference Control Value and a Reference Breast Cancer Value comprises a mean concentration value of each protein in saliva from a group of individuals with breast cancer; and determining a diagnosis of either increased or decreased risk of breast cancer for the patient based on a result of the comparison. In some embodiments, the breast cancer comprises ductal carcinoma in situ (DCIS).

In some embodiments, comparing the test data yields a comparison result in which the concentration value of at least the first protein marker differs from the Reference Control Value of the respective protein marker, wherein the difference is significant at a level in the range of p<0.05 to p<0.0001.

In some embodiments, wherein the Reference Control Value comprises a mean concentration value of each protein marker in saliva from a group of breast cancer-free individuals, and the Reference Cancer Value comprises a mean concentration value of each protein in saliva from a group of individuals with cancer.

In some embodiments, at least a second protein marker is also included which is selected from the group consisting of cytochrome p450, ubiquitin, carbonic anhydrase VI (CAH6), cytokeratin 4 (K2C4), cystatin A (CYTA), epidermal fatty acid-binding protein (FABP4), Ig gamma-1 chain C region (IGHGI), lactoferrin (TRFL), bact. perm.-increasing protein-1 (BPIL1), haptoglobin (HPT), profilin-1 (PROF1) and zinc-alpha-2-glycoprotein (ZA2G). A diagnosis of either increased or decreased risk of breast cancer for the patient is determined based on a result of the comparison.

In some embodiments, wherein at least one protein marker is known to be differentially expressed among benign breast disease (fibroadenoma), breast cancer and tumor-free breast tissue; a mean concentration value of each protein marker in saliva from a group of individuals with benign breast tumor (Reference Benign Value) is known, and differentiating increased risk of cancer (e.g., DCIS) from increased risk of benign fibroadenoma in the patient includes comparing the measured values to the Reference Benign Value.

In some embodiments, wherein the Reference Cancer Value and/or the Reference Benign Value differs from the Reference Control Value, wherein the difference is significant at a level in the range of p<0.05 to p<0.0001.

In some embodiments, a second protein marker is included which is selected from the group consisting of cytochrome p450, ubiquitin, alpha enolase (ENOA), Ig alpha-2 chain C region (IGHA2), interleukin-1 receptor antagonist protein precursor (IL-1RA), S100 calcium-binding protein A7 (S100A7), short palate, lung and nasal epithelial cancer associated protein 2 (SPLC2), and HER2/neu. In this case, determining a diagnosis of increased or decreased likelihood of fibroadenoma in the patient is based on a result of the above-described comparisons.

In some embodiments, the reference panel is prepared by a process that comprises isotopic labeling of salivary proteins and subjecting the labeled proteins to liquid chromatography tandem mass spectrometry to qualitatively and quantitatively characterize salivary proteins of said respective cancer and control groups, and then determining from the characterization a mean concentration value for each protein marker in the respective breast cancer and control groups.

In some embodiments, an above-described method includes obtaining a second saliva sample from the patient subsequent to the first sample; measuring in the second saliva sample a concentration of at least the first protein marker, to provide a second set of test data comprising a second concentration value of each protein marker in the saliva sample; comparing the second set of test data to the reference panel; and determining a second diagnosis of either increased or decreased risk of breast cancer in the patient based on a result of an above-described comparison. In some embodiments, a second set of test data is compared to a first set of test data to determine whether a difference in the concentration value of at least the first protein marker exists.

In some embodiments, the first saliva sample is obtained prior to surgical removal of cancerous breast tissue from the patient. In some embodiments, the patient has received therapeutic treatment for breast cancer prior to obtaining a second saliva sample. In some embodiments of an above-described method, determining a diagnosis comprises an indication whether the therapeutic treatment is effective in the patient.

In accordance with certain embodiments, a method of screening a population for increased risk of breast cancer, comprises measuring in saliva samples from respective patients a concentration of at least a first protein marker, wherein the first protein marker is either ubiquitin or cytochrome p450, to provide a set of test data comprising a concentration value of each protein marker in each patient's saliva sample; comparing each set of test data to a reference panel comprising a Reference Control Value and a Reference Cancer Value; and determining a diagnosis of either increased or decreased risk of breast cancer for each patient, based on a result of a respective comparison; and administering a therapeutic treatment to patients with a diagnosis of increased risk of breast cancer, based on a result of the respective comparison.

In accordance with certain embodiments, a test kit for identifying a person at increased risk of breast cancer, comprises a first set of components for performing a first immunoassay to detect and quantify a first protein marker selected from the group consisting of cytochrome p450 and ubiquitin in saliva; and at least a second set of components for performing at least a second immunoassay to detect and quantify at least one additional protein marker selected from the group consisting of cytochrome p450, ubiquitin, carbonic anhydrase VI (CAH6), cytokeratin 4 (K2C4), cystatin A (CYTA), epidermal fatty acid-binding protein (FABP4), Ig gamma-1 chain C region (IGHGI), lactoferrin (TRFL), bacterial permeability-increasing protein-1 (BPIL1), haptoglobin (HPT), profilin-1 (PROF1) and zinc-alpha-2-glycoprotein (ZA2G).

In some embodiments, an above-described test kit may include a second set of components for performing a third immunoassay to detect and quantify at least one additional protein marker selected from the group consisting of alpha enolase (ENOA), Ig alpha-2 chain C region (IGHA2), interleukin-1 receptor antagonist protein precursor (IL-1RA), S100 calcium-binding protein A7 (S10A7) and short palate, lung and nasal epithelial cancer associated protein 2 (SPLC2) and HER2/neu.

In some embodiments, a test kit may further comprise at least one control solution containing a protein or peptide to serve as a positive or negative control for each respective immunoassay. These and other embodiments and features will be apparent from the detailed description and drawings.

### BRIEF DESCRIPTION OF THE DRAWINGS

FIG. 1 is a set of Venn diagrams showing overlapping proteins between three groups of women, in accordance with an exemplary set of data illustrating an embodiment of the invention.

FIG. 2 is a chart showing the percentages of the 130 proteins from FIG. 1 that correspond to respective functions.

FIG. 3 is a chart showing differential protein expression in cancer saliva (darker bars) and benign breast disease saliva (lighter bars) versus normal control represented by the 0 position.

FIG. 4 is a chart showing differential protein expression in breast cancer saliva (bars) versus benign saliva represented by the 0 position.

FIG. 5 is a box flow diagram illustrating steps of an iTRAQ™ proteomic procedure. ESI, electrospray; HPLC, high performance liquid chromatography; TOF, time of flight.

FIG. 6 is a process flow diagram illustrating the analytical strategy of comparisons associated with the iTRAQ™ procedure of FIG. 5.

FIG. 7 illustrates up-regulated growth pathways associated with the salivary protein profile in the presence of carcinoma of the breast, showing expression of cancer genes in tumor saliva vs. control saliva.

FIG. 8 illustrates down-regulated apoptotic pathways associated with the salivary protein profile in the presence of carcinoma of the breast, showing expression of cancer genes in tumor saliva vs. control saliva.

FIG. 9 is a western blot illustrating the presence of cytochrome p450 in human salivary gland cell lysate, SKBR3 cell lysate and saliva.

FIG. 10. is a western blot of a SKBR3 cell lysate control dilution series with cytochrome p450 antibody.

FIG. 11 is a western blot obtained with control (stage IV breast cancer), healthy and benign breast disease salivas, and with salivas from stages 0, I, IIa and III breast cancer groups using anti-cytochrome p450 antibody.

FIG. 12 shows the results of a ROC curve analysis of the candidate biomarker cytochrome p450 using 2-step indirect ELISA. Data from benign breast disease vs. breast cancer (stages 0 to IV) obtained with cytochrome p450 are shown.

FIG. 13 shows the results of a ROC curve analysis of the candidate biomarker ubiquitin using 2-step indirect ELISA. Data from benign breast disease vs. breast cancer (stages 0 to IV) obtained with ubiquitin are shown.

FIG. 14 shows the results of a ROC curve analysis of the candidate biomarker zinc-alpha-2-glycoprotein using 2-step indirect ELISA. Data from benign breast disease vs. breast cancer (stages 0 to IV) obtained with zinc-alpha-2-glycoprotein are shown.

FIG. 15 shows the results of a ROC curve analysis of the candidate biomarker cytochrome p450 using 2-step indirect ELISA. Data from healthy vs. benign breast disease saliva samples obtained with cytochrome p450 are shown.

FIG. 16 shows the results of a ROC curve analysis of the candidate biomarker ubiquitin using 2-step indirect ELISA. Data from healthy vs. benign breast disease saliva samples obtained with ubiquitin are shown.

FIG. 17 shows the results of a ROC curve analysis of the candidate biomarker zinc-alpha-2-glycoprotein using 2-step indirect ELISA. Data from healthy vs. benign breast disease saliva samples obtained with zinc-alpha-2-glycoprotein are shown.

### DEFINITIONS

In the following discussion and in the claims, the terms “comprising,” “including” and “containing” are used in an open-ended fashion, and thus should be interpreted to mean “including, but not limited to . . . ”.

The singular forms “a,” “an,” and “the” include plural referents unless the context clearly dictates otherwise.

The term “about,” when used in the context of a numerical value, means approximately or reasonably close to the given number, and generally includes, but is not limited to, ±10% of the stated number.

The term “secondary to carcinoma of the breast,” when referring to one or more up-regulated or down-regulated proteins, means resulting from metabolic or regulatory effects on the other tissues, fluids or structures due to carcinoma of the breast.

The term “salivary proteome” refers to the complement of proteins and peptides expressed in the saliva of a patient at a particular time and under given conditions.

The term “concentration value” refers to a quantitative amount or any other appropriate indication of a concentration, such as, for example, a colorimetric indicator score (e.g., +/−).

The terms “protein marker” and “biomarker” are used interchangeably herein. It should be understood that a protein marker does not necessarily require the full amino acid sequence of the protein in the diagnostic methods described herein, but in many cases will consist of a conserved portion or fragment of such protein sufficient to serve as a representative marker for the intact protein adequate to serve as an epitope for antibody recognition.

The term “epitope” refers to any polypeptide determinant capable of selectively binding to an immunoglobulin or a T-cell receptor. In general, an epitope is a region of an antigen that is selectively bound by an antibody. In certain cases, an epitope may include chemically active surface groupings of molecules such as amino acids, sugar side chains, phosphoryl, and/or sulfonyl groups. Additionally, an epitope may have specific three dimensional structural characteristics (e.g., a “conformational” epitope) and/or specific charge characteristics. An epitope is defined as “the same” as another epitope if a particular antibody selectively binds to both epitopes. In certain cases, polypeptides having different primary amino acid sequences may comprise epitopes that are the same, and epitopes that are the same may have different primary amino acid sequences. Different antibodies are said to bind to the same epitope if they compete for selective binding to that epitope.

The term “increased risk” of breast cancer refers to a greater likelihood of a patient's having existing breast cancer, or of developing breast cancer. In some instances, breast cancer comprises ductal carcinoma in situ (DCIS). In some instances, increased risk of breast cancer is distinguishable from increased risk of benign breast disease or fibroadenoma (benign tumor) of the breast.

In the context of determining increased risk of breast cancer or benign tumor, a “significant” difference between protein marker levels in saliva generally refers to a P value in the range of p<0.001 to p<0.0001 levels.

### DETAILED DESCRIPTION

It was investigated in the present studies whether protein-by-products secondary to cancer related oncogenes that are over or under expressed appear in the saliva of breast cancer patients. It is proposed that saliva is a fluid suffused with solubilized protein by-products of oncogenic expression and these proteins are modulated secondary to ductal carcinoma in situ (DCIS) of the breast. Additionally, there are salivary protein profiles that are unique to both DCIS and fibroadenoma tumors. Such differences between DCIS and fibroadenoma are potentially valuable for noninvasively detecting and diagnosing breast cancer.

Saliva was selected for investigation as a diagnostic fluid primarily for two reasons: 1) collection of saliva is a non-invasive procedure that can be conducted in any environment requiring no special skills or equipment; and 2) the physiology of the oral cavity is such that the flow of secreted fluid is continually flushing and refreshing the fluid content of the mouth. Therefore, the composition of the fluid at any moment temporally reflects the metabolic activity of the secretory elements generating that fluid. There are also significant potential advantages over the study of plasma. In plasma the concentration of proteins can vary over nine orders of magnitude, which severely diminishes the likelihood of detecting those proteins at the lower end of the scale. The second consideration is that blood is composed of peptides, proteins and cells that have half lives ranging from seconds to weeks or even a month or more. As a consequence, the presence of a given substance might not accurately reflect the current state of the organism.

Protein profiling was performed on three pooled, stimulated whole saliva specimens. One specimen consisted of pooled saliva from 10 healthy subjects, another specimen was a pooled saliva specimen from 10 benign tumor patients (fibroadenomas), and the third specimen was from subjects diagnosed with ductal carcinoma in situ (DCIS). Fibroadenoma was selected due to its high prevalence among benign breast tumors. For some of the studies described herein, DCIS was selected as this represents the lowest detectable tumor load according to the AJCC Cancer Staging Handbook, Part VII Breast (Greene, et al. editors. AJCC CANCER STAGING HANDBOOK. 6th ed. New York: Springer-Verlag; 2001). The cancer cohort, internally, was estrogen, progesterone and Her2/neu receptor status negative as determined by the pathology report. All subjects were matched for age and race and were non-tobacco users.

Saliva Collection and Sample Preparation.

Stimulated whole salivary gland secretion is based on the reflex response occurring during the mastication of a bolus of food. Usually, a standardized bolus (1 gram) of paraffin or a gum base (generously provided by the Wrigley Co., Peoria, Ill.) is given to the subject to chew at a regular rate. The individual, upon sufficient accumulation of saliva in the oral cavity, expectorates periodically into a preweighed disposable plastic cup. This procedure is continued for a period of five minutes. The volume and flow rate is then recorded along with a brief description of the specimen's physical appearance, similar to the procedure described by Berkhed and Heintze (in: Tenovuo, J. O., editor. HUMAN SALIVA: CLINICAL CHEMISTRY AND MICROBIOLOGY—VOLUME 1. Boca Raton: CRC press; 1989). The cup with the saliva specimen is reweighed and the flow rate determined gravimetrically. This salivary collection method may be modified for more consistent protein analyses as described by Streckfus, et al. (Oral Surg Oral Med Oral Path. 2001, 91(2), 174-180). A protease inhibitor from Sigma Co (St. Louis, Mich., USA) is added along with enough orthovanadate from a 100 mM stock solution to bring its concentration to 1 mM. The treated samples are centrifuged for 10 minutes at approximately 15,000×g in a conventional table top centrifuge. The supernatant is divided into 1 ml aliquots and frozen at −80° C.

C-MS/MS Mass Spectroscopy with Isotopic Labeling.

Mass spectrometry, liquid chromatography, analytical software and bioinformatics techniques are used to analyze complex salivary peptide mixtures, wherein such techniques are capable of detecting differences in abundance of a given protein of over 8 orders of magnitude, as described by Wilmarth, et al. (J Prot Res. 2004, 3, 1017-23). For example, isotopic labeling coupled with liquid chromatography tandem mass spectrometry (IL-LC-MS/MS) to characterize the salivary proteome is employed as described by Gu, et al. (Mol Cell Proteomics. 2004, 10, 998-1008). The preferred method is a mass spectroscopy based method that uses isotope coding of complex protein mixtures such as tissue extracts, blood, urine or saliva to identify differentially expressed proteins, according to the method of Shevchenko, et al. (Mol Biotech. 2002, 20, 107-18). In this way, changes in the level of expression of a protein are readily identified, thus permitting the analysis of putative regulatory pathways and providing information regarding the pathological disturbances in addition to potential biomarkers of disease. In embodiments, the analysis is performed on a tandem QqTOF QStar™ XL mass spectrometer (Applied Biosystems, Foster City, Calif., USA) equipped with an LC Packings (Sunnyvale, Calif., USA) HPLC for capillary chromatography. The HPLC is coupled to the mass spectrometer by a nanospray ESI head (Protana, Odense, Denmark) for maximal sensitivity, as described by Shevchenko, et al. (id). An advantage of tandem mass spectrometry combined with LC is enhanced sensitivity and the peptide separations afforded by chromatography. Thus, even in complex protein mixtures, MS/MS data can be used to sequence and identify peptides by sequence analysis with a high degree of confidence.

Isotopic labeling of protein mixtures has proven to be a useful technique for the analysis of relative expression levels of proteins in complex protein mixtures such as plasma, saliva urine or cell extracts. There are numerous methods that are based on isotopically labeled protein modifying reagents to label or tag proteins to determine relative or absolute concentrations in complex mixtures. The higher resolution offered by the tandem Qq-TOF mass spectrometer is ideally suited to isotopically labeled applications.

There are two methods that are based on isotopically labeled protein modifying reagents to label or tag proteins in the mixtures: the iCAT and the iTRAQ™ techniques. The general approach for both techniques is to label two to four different saliva samples with agents that are chemically identical, but have different atomic masses. Any chemically based purification technique does not distinguish between the two; however, in the mass spectrometer they can be distinguished by their difference in atomic mass. Because they are chemically identical, they ionize with the same efficiency in the mass spectrometer permitting an estimate of their relative concentrations based on the relative peak intensities. An iCAT procedure uses cysteine-specific labels that include a biotin moiety in their structure. An avidan binding step enables a high degree of enrichment of iCAT-labeled peptides.

The recently introduced iTRAQ™ reagents (Applied Biosystems) are amino reactive compounds that are used to label peptides in a total protein digest of a fluid. The tag remains intact through TOF-MS analysis; however, it is revealed during collision induced dissociation by MSMS analysis. Thus, in the MSMS spectrum for each peptide there is a fingerprint indicating the amount of that peptide from each of the different protein pools. Since virtually all of the peptides in a mixture are labeled by the reaction, numerous proteins in complex mixtures are identified and can be compared for their relative concentrations in each mixture. Thus even in complex mixtures there is a high degree of confidence in the identification.

### Antibodies to Protein Markers

Antibodies that selectively bind to one or more epitopes of the protein markers disclosed herein, or to epitopes of conserved variants, are used in some embodiments of the immunoassays and methods described herein. For example, in some embodiments antibodies to cytochrome p450 or ubiquitin or epitopes of conserved variants of cytochrome p450 or ubiquitin and its fragments are used in immunoassays described herein. Antibodies suitable for use in these immunoassays in some cases include, but are not limited to, those available commercially from, Abcam Inc., (Cambridge, Mass.), Abnova (Taipei, Taiwan), AbD Serotec (a division of MorphoSys US Inc. Raleigh, N.C.), ABR-Affinity Bioreagents (now Thermo Fisher Scientific-Rockford, Ill.), Santa Cruz Biotechnology (Santa Cruz, Calif.), DAKO (Carpinteria, Calif.), GenWay Biotech (San Diego, Calif.) and BD Biosciences (San Jose, Calif.), for example.

Non-limiting examples of such antibodies include polyclonal antibodies, monoclonal antibodies (mAbs), humanized antibodies, human-engineered antibodies, fully human antibodies, chimeric antibodies, single chain antibodies, Fab fragments, F(ab′)2 fragments, fragments produced by a Fab expression library, anti-idiotypic (anti-Id) antibodies, catalytic antibodies, and epitope-binding fragments of any of the above. In some applications, the antibodies, or fragments thereof, will preferentially bind to an above-identified protein marker (e.g., cytochrome p450 or ubiquitin), as opposed to other related proteins. In those cases, the antibodies, or fragments thereof, selectively bind to the protein marker with a higher affinity or avidity than they bind to other related proteins.

An antibody “selectively binds” an antigen when it preferentially recognizes the antigen in a complex mixture of proteins and/or other macromolecules. The antibodies employed in some of the methods disclosed herein comprise an antigen-binding site that selectively binds to a particular epitope. Such antibodies can be capable of binding to different antigens, so long as the different antigens comprise that particular epitope. In some applications, homologous proteins from different species comprise the same epitope. In various applications, an antibody selectively binds an antigen when the dissociation constant (K_{D}) is 1 μM, or when the dissociation constant is 100 nM, or when the dissociation constant is 10 nM, for example.

Epitopes may be identified from primary amino acid sequences on the basis of hydrophilicity. These regions are also referred to as “epitopic core regions.” In general, cytochrome p450 or ubiquitin peptides selected for immunizing an animal comprise one or more epitopes, as such peptides are likely to be immunogenic. In general, peptide immunogens and epitopes are those that are predicted to be hydrophilic and/or likely to be exposed on the surface of native cytochrome p450 or ubiquitin in its folded state. In certain embodiments, peptide segments that are predicted to form β□-turns, and are therefore likely to be exposed on the surface of a protein, may be selected as immunogens. Alternatively, it is not necessary that the epitope be expressed on the surface of the protein. Many immunological techniques utilize the addition of reagents to facilitate protein unfolding, thereby unmasking epitopes that were unavailable prior to the manipulation. Guidance for selecting suitable immunogenic peptides and related techniques are provided, for example, in “Current Protocols in Molecular Biology,” supra, Ch. 11.14, and “Antibodies: A Laboratory Manual,” supra, Ch. 5.

Certain algorithms are known to those skilled in the art for predicting whether a peptide segment of a protein is hydrophilic, and therefore likely to be exposed on the surface of the protein. These algorithms use the primary sequence information of a protein to make such predictions, and are based on the method of, for example, Hopp and Woods, Proc. Natl. Acad. Sci. USA 78:3824-3828, 1981, or Kyte and Doolittle, supra. Certain exemplary algorithms are known to those skilled in the art for predicting the secondary structure of a protein based on the primary amino acid sequence of the protein (see, e.g., Corrigan and Huang, Comput. Programs Biomed 15:163-168, 1982, Chou and Fasman, Ann. Rev. Biochem. 47:251-276, 1978, Moult, Curr. Opin. Biotechnol. 7:422-427, 1996, Chou and Fasman, Biochemistry 13:222-245, 1974, Chou and Fasman, Biochemistry 13:211-222, 1974, Chou and Fasman, Adv. Enzymol. Relat. Areas Mol. Biol. 47:45-148, 1978, and Chou and Fasman, Biophys. J. 26:367-383, 1979). Computer programs are currently available to assist with predicting secondary structure. One method of predicting secondary structure is based upon homology modeling. For example, two polypeptides or proteins that have a sequence identity of greater than 30%, or similarity greater than 40%, often have similar structural topologies. The growth of the Protein Structural Database (PSDB); Berman et al., Nucleic Acids Res. 28:235-242, 2000) and the Protein Data Bank (PDB) has provided enhanced predictability of secondary structure, including the potential number of folds within the structure of a polypeptide (see, e.g., Holm and Sander, Nucleic Acids Res. 27:244-247, 1999). It has been suggested there are a limited number of folds in a given polypeptide or protein, and once a critical number of structures have been resolved, structural prediction will become much more accurate (Brenner, et al., Curr. Opin. Struct. Biol. 7:369-376, 1997). Additional methods of predicting secondary structure include “threading” (see, e.g., Jones, Curr. Opin. Struct. Biol. 7:377-387, 1997, and Sippl and Flockner, Structure 4:15-19, 1996), “profile analysis” (see, e.g., Bowie et al., Science 253:164-170, 1991, Gribskov, et al. Meth. Enzymol. 183:146-159, 1990, and Gribskov, et al. Proc. Natl. Acad. Sci. USA 84:4355-4358, 1987), and “evolutionary linkage” (see, e.g., Holm and Sander, 1999, supra, and Brenner et al., 1997, supra).

Antibodies that selectively bind to cytochrome p450 or ubiquitin may be used, for example, in the detection and determination of cytochrome p450 or ubiquitin levels in a biological sample, preferably saliva and may, therefore, be utilized as part of a diagnostic or prognostic technique whereby patients may be tested to determine if normal or abnormal amounts of cytochrome p450 or ubiquitin are present. In some embodiments, these antibodies are used in the cytochrome p450 or ubiquitin immunoassays described herein, and in the identification and quantitation of the level of cytochrome p450 or ubiquitin in the bodily fluids, preferably saliva, of patients with disorders associated with breast cancer in order to identify those at increased risk of developing breast cancer.

A native antibody typically has a tetrameric structure comprising two identical pairs of polypeptide chains, each pair having one light chain (typically about 25 kDa) and one heavy chain (typically about 50-70 kDa). In a native antibody, a heavy chain comprises a variable region, V_{H}, and three constant regions, C_{H}1, C_{H}2, and C_{H}3. The V_{H} domain is at the amino-terminus of the heavy chain, and the C_{H}3 domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In humans, for example, native human light chains are typically classified as kappa and lambda light chains. Native human heavy chains are typically classified as mu, delta, gamma, alpha, or epsilon, and define the isotype of the antibody as IgM, IgD, IgG, IgA, and IgE, respectively. IgG has subclasses, including, but not limited to, IgG1, IgG2, IgG3, and IgG4. IgM has subclasses including, but not limited to, IgM1 and IgM2. IgA has subclasses including, but not limited to, IgA1 and IgA2. Within native human light and heavy chains, the variable and constant regions are typically joined by a “J” region of about 12 or more amino acids, with the heavy chain also including a “D” region of about 10 more amino acids (“Fundamental Immunology”, 2^{nd} Ed. Ch. 7 (Paul, ed., Raven Press, New York, N.Y., 1989)). In various applications, the antibodies used in an immunoassay are of any of the isotypes or isotype subclasses set forth above.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen binding site. For example, H3, in certain instances, can be as short as two amino acid residues or greater than 26. The assignment of amino acids to each domain is typically in accordance with the definitions in “Sequences of Proteins of Immunological Interest” (Kabat, et al, eds., National Institutes of Health, Publication No. 91-3242, 5^{th} Ed., United States Department of Health and Human Services, Bethesda, Md., 1991), Chothia and Lesk, J. Mol. Biol. 196:901-917, 1987, or Chothia et al., Nature 342:878-883, 1989. In the present application, the term “CDR” refers to a CDR from either the light or heavy chain, unless otherwise specified.

Production of Cytochrome p450 or Ubiquitin Antibodies

In addition to cytochrome p450 or ubiquitin antibodies and cytochrome p450 or ubiquitin assay kits as described herein, or those that are known to those of skill in the art and may be commercially available, antibodies for use in the various immunoassays disclosed herein also include those that are generated de novo. For the production of antibodies, various host animals, including but not limited to chickens, hamsters, guinea pigs, rabbits, sheep, goats, horses, may be immunized by injection with a cytochrome p450 or ubiquitin protein, polypeptide, or peptide, a truncated cytochrome p450 or ubiquitin polypeptide, a functional equivalent of cytochrome p450 or ubiquitin, a mutant of cytochrome p450 or ubiquitin, an antigenic fragment thereof, or combinations thereof. Such host animals may include, but are not limited to, rabbits, mice, and rats, and cytochrome p450 or ubiquitin “knock-out” variants of the same. In addition, antibodies can be produced by immunizing female birds (chickens, for example) and harvesting the IgY antibodies present in their eggs. Various adjuvants may be used to increase the immunological response, depending on the host species, including, but not limited to, Freund's adjuvant (complete and incomplete), mineral salts such as aluminum hydroxide or aluminum phosphate, surface active substances, chitosan, lysolecithin, pluronic polyols, polyanions, peptides, oil emulsions, and potentially useful human adjuvants such as BCG (Bacille Calmette-Guerin) and Corynebacterium parvum. Alternatively, the immune response could be enhanced by combination and/or coupling with molecules such as keyhole limpet hemocyanin (KLH), tetanus toxoid, diphtheria toxoid, ovalbumin, cholera toxin, or fragments thereof. Alternatively expression as a fusion protein, such as, but not limited to, GST, His6, etc. can also be used.

Polyclonal antibodies are heterogeneous populations of antibody molecules, such as those derived from the sera of the immunized animals or by mixing B-cells or monoclonal antibodies. Monoclonal antibodies, which are homogeneous populations of antibodies that arise from a single B-cell or its which selectively bind to a particular antigen or epitope, may be obtained by any technique that provides for the production of antibody molecules by continuous cell lines in culture. These include, but are not limited to, the hybridoma technique (Kohler and Milstein, Nature 256:495-497, 1975, U.S. Pat. No. 4,376,110, and “Antibodies: A Laboratory Manual,” supra, Ch. 6), the human B-cell hybridoma technique (Kozbor and Roder, Immunol. Today 4:72-79, 1983, and Cote et al., Proc. Natl. Acad. Sci. USA 80:2026-2030, 1983), and the EBV-hybridoma technique (Cole et al., Mol. Cell. Biochem. 62:109-120, 1984, and Cole et al., Cancer Res. 44:2750-2753, 1984). A suitable animal, such as a mouse, rat, hamster, monkey, or other mammal, or an avian species, is immunized with an immunogen to produce antibody-secreting cells, including, but not limited to, B-cells, such as lymphocytes or splenocytes. In certain embodiments, lymphocytes (e.g., human lymphocytes) are immunized in vitro to generate antibody-secreting cells (Borrebaeck et al., Proc. Natl. Acad. Sci. USA 85:3995-3999, 1988). The hybridomas producing the monoclonal antibodies that are used in certain embodiments may be cultivated in vitro or in vivo. In some instances, the production of high titer monoclonal antibodies in vivo is the preferred method of producing antibodies for use in a testing method described herein.

For some applications, antibody-secreting cells are fused with an “immortalized” cell line, such as a myeloid-type cell line, to produce hybridoma cells. Hybridoma cells that produce the desired antibodies can be identified, for example, by ELISA, and can then be subcloned and cultured using standard methods, or grown in vivo as ascites tumors in a suitable animal host. For some applications, monoclonal antibodies are isolated from hybridoma culture medium, serum, or ascites fluid using standard separation procedures, such as affinity chromatography (see, e.g., “Antibodies: A Laboratory Manual,” supra, Ch. 8).

For assaying cytochrome p450 or ubiquitin levels, in some cases high affinity antibodies are generated using animals that have been genetically engineered to be deficient in cytochrome p450 or ubiquitin production and activity. An example of such knock-out animals (mice) are produced using established gene trapping methods, and viable animals that are genetically homozygous for the genetically engineered cytochrome p450 or ubiquitin mutation are generated and characterized. Given the relatedness of mammalian cytochrome p450 or ubiquitin amino acid sequences, the presently described homozygous knock-out mice (having never seen, and thus never been tolerized to, cytochrome p450 or ubiquitin) can be advantageously applied to the generation of antibodies against mammalian cytochrome p450 or ubiquitin sequences (i.e., cytochrome p450 or ubiquitin will be immunogenic in cytochrome p450 or ubiquitin homozygous knock-out animals). High affinity anti-cytochrome p450 or ubiquitin antibodies generated from such animals can be formulated into immunoassays that are used, as described herein, to identify those patients at risk for breast cancer.

For example, human monoclonal antibodies are raised in transgenic animals (e.g., mice) that are capable of producing human antibodies (see, e.g., U.S. Pat. Nos. 6,075,181 and 6,114,598, and PCT Patent Application Publication No. WO 1998/24893). For example, human immunoglobulin genes can be introduced (e.g., using yeast artificial chromosomes, human chromosome fragments, or germline integration) into mice in which the endogenous Ig genes have been inactivated (see, e.g., Jakobovits et al., Nature 362:255-258, 1993, Tomizuka et al., Proc. Natl. Acad. Sci. USA 97:722-727, 2000, and Mendez et al., Nat. Genet. 15:146-156, 1997, describing the XenoMouse II® line of transgenic mice). Additional exemplary methods and transgenic mice suitable for the production of human monoclonal antibodies are described, e.g., in Jakobovits, Curr. Opin. Biotechnol. 6:561-566, 1995, Lonberg and Hiuszar, Int. Rev. Immunol. 13:65-93, 1995, Fishwild et al., Nat. Biotechnol. 14:845-851, 1996, Green, J. Immunol. Methods 231:11-23, 1999, and Little et al., Immunol. Today 21:364-370, 2000.

In addition, techniques developed for the production of “chimeric antibodies” (Morrison et al., Proc. Natl. Acad. Sci. USA 81:6851-6855, 1984, Neuberger et al., Nature 312:604-608, 1984, and Takeda et al., Nature 314:452-454, 1985), for example by splicing the genes from a mouse antibody molecule of appropriate antigen selectivity together with genes from a human antibody molecule of appropriate biological activity, can be used. A chimeric antibody is a molecule in which different portions are derived from different animal species, such as those having a variable region derived from a murine monoclonal antibody and a human immunoglobulin constant region. Such technologies are described in, for example, U.S. Pat. Nos. 6,075,181 and 5,877,397.

Monoclonal antibodies that are employed in some applications for determining cytochrome p450 or ubiquitin levels can also be produced by recombinant techniques (see, e.g., U.S. Pat. No. 4,816,567). In such embodiments, nucleic acids encoding monoclonal antibody chains are cloned and expressed in a suitable host cell. For example, RNA can be prepared from cells expressing the desired antibody, such as mature B-cells or hybridoma cells, which can then be used to make cDNA, using standard methods. The cDNA encoding a heavy or light chain polypeptide can be amplified, for example, by PCR, using specific oligonucleotide primers. The cDNA can then be cloned into a suitable expression vector, which is then transformed or transfected into a suitable host cell, such as a host cell that does not endogenously produce antibody.

Transformation or transfection may be by any known method for introducing polynucleotides into a host cell. Certain exemplary methods include, but are not limited to, packaging the polynucleotide in a virus (or into a viral vector) and transducing a host cell with the virus (or vector) and using certain transfection procedures known in the art, as exemplified by U.S. Pat. Nos. 4,399,216, 4,912,040, 4,740,461, and 4,959,455. In certain embodiments, the transformation procedure used may depend upon the host to be transformed. Various methods for introduction of heterologous polynucleotides into mammalian cells are known in the art and include, but are not limited to, dextran-mediated transfection, calcium phosphate precipitation, polybrene-mediated transfection, protoplast fusion, electroporation, encapsulation of the polynucleotide(s) in liposomes, and direct microinjection of the DNA into nuclei. In embodiments where heavy and light chains are co-expressed in the same host, reconstituted antibody may be isolated.

Alternatively, techniques described for the production of single chain antibodies (Bird et al., Science 242:423-426, 1988, Huston et al., Proc. Natl. Acad. Sci. USA 85:5879-5883, 1988, Ward et al., Nature 341:544-546, 1989, PCT Patent Application Publication No. WO 1988/01649, and U.S. Pat. Nos. 4,946,778 and 5,260,203) can be adapted to produce single chain antibodies against cytochrome p450 or ubiquitin gene products or epitopes. Single chain antibodies are formed by linking the heavy and light chain fragments of the Fv region via an amino acid bridge, resulting in a single chain polypeptide with an antigen binding region.

In some applications, a method or test kit disclosed herein for measuring cytochrome p450 or ubiquitin levels employs antibody fragments, including, but not limited to, Fab, Fab′, F(ab′)₂, Fv, scFv, Fd, diabodies, and other antibody fragments that retain at least a portion of the variable region of an intact antibody (see, e.g., Hudson and Souriau, Nature Med. 9:129-134, 2003). A Fab fragment comprises one light chain and the C_{H}1 and variable region of one heavy chain. The heavy chain of a Fab molecule cannot form a disulfide bond with another heavy chain molecule. A Fab′ fragment comprises one light chain and one heavy chain that comprises additional constant region, extending between the C_{H}1 and C_{H}2 domains, and can be generated by reducing the disulfide bridges of F(ab′)₂ fragments. An interchain disulfide bond can be formed between two heavy chains of a Fab′ fragment to form a F(ab′)₂ molecule, which can be produced by pepsin digestion of an antibody molecule. A Fv fragment comprises the variable regions from both the heavy and light chains, but lacks the constant regions. In certain instances, a single variable region (one-half of a Fv) may have the ability to recognize and bind antigen, albeit with lower affinity than the Fv. A Fab expression library may also be constructed (Huse et al., Science 246:1275-1281, 1989) to allow rapid and easy identification of monoclonal Fab fragments with the desired selectivity.

Monoclonal antibodies employed in certain embodiments may also be produced using a display-based method. For example, monoclonal antibodies can be produced using phage display techniques (see, e.g., Hoogenboom, Method Mol. Biol. 178:1-37, 2002, Clackson et al., Nature 352:624-628, 1991, and Marks et al., J. Mol. Biol. 222:581-597, 1991). For example, a library of antibodies can be displayed on the surface of a filamentous phage, such as the nonlytic filamentous phage fd or M13. The antibodies can be antibody fragments, such as scFvs, Fabs, Fvs with an engineered intermolecular disulfide bond to stabilize the V_{H}-V_{L} pair, and diabodies. Using these techniques, antibodies with the desired binding selectivity can then be selected.

For example, in some instances, variable gene repertoires are prepared by PCR amplification of genomic DNA or cDNA derived from the mRNA of antibody-secreting cells, such as B-cells. For example, cDNA encoding the variable regions of heavy and light chains can be amplified by PCR, and the heavy chain cDNA and light chain cDNA cloned into a suitable vector. The heavy chain cDNA and light chain cDNA can be randomly combined during the cloning process, thereby resulting in the assembly of a cDNA library encoding diverse scFvs or Fabs. Alternatively, the heavy chain cDNA and light chain cDNA can be ligated, for example by stepwise cloning, before being cloned into a suitable vector.

Suitable vectors include, but are not limited to, phage display vectors, such as a phagemid vectors. Certain exemplary phagemid vectors, such as pCES1, are known to those skilled in the art. In certain embodiments, cDNA encoding both heavy and light chains is present on the same vector. For example, cDNA encoding scFvs can be cloned in-frame with all or a portion of gene III, which encodes the minor phage coat protein pill. The phagemid then directs the expression of the scFv-pIII fusion on the phage surface. Alternatively, cDNA encoding heavy chain (or light chain) can be cloned in-frame with all or a portion of gene III, and cDNA encoding light chain (or heavy chain) can be cloned downstream of a signal sequence in the same vector. The signal sequence directs expression of the light chain (or heavy chain) into the periplasm of the host cell, where the heavy and light chains assemble into Fab fragments. In other methods, cDNA encoding heavy chain and cDNA encoding light chain can be present on separate vectors. In these methods, heavy chain and light chain cDNA are cloned separately, one into a phagemid and the other into a phage vector, which both contain signals for in vivo recombination in the host cell. The recombinant phagemid and/or phage vectors are introduced into a suitable bacterial host, such as E. coli. When using certain phagemids, the host can be infected with helper phage to supply phage structural proteins, thereby allowing expression of phage particles carrying the antibody-pIII fusion protein on the phage surface.

“Synthetic” antibody libraries can be constructed using repertoires of variable genes that are rearranged in vitro. For example, individual gene segments encoding heavy or light chains (V-D-J or V-J, respectively) are randomly combined using PCR. Additional sequence diversity can be introduced into the CDRs, such as CDR3 (H3 of the heavy chain), and possibly FRs, by error prone PCR.

“Naïve” or “universal” phage display libraries can be constructed, as described above, using nucleic acids from a naïve (unimnmunized) animal, while “immunized” phage display libraries can be constructed, as described above, using nucleic acids from an immunized animal. Exemplary universal human antibody phage display libraries are available from commercial sources, and include, but are not limited to, the HuCAL® series of libraries from MorphoSys AG (Martinstried/Planegg, Germany), libraries from Crucell (Leiden, the Netherlands) using MAbstract® technology, the n-CoDeR™ Fab library from BioInvent international AB (Lund, Sweden), and libraries available from Cambridge Antibody Technology (Cambridge, United Kingdom).

Selection of antibodies having the desired binding selectivity from a phage display library can be achieved by successive panning steps. In panning, library phage preparations are exposed to one or more antigen(s), such as one or more cytochrome p450 or ubiquitin antigen(s). The phage-antigen complexes are then washed, and unbound phage are discarded. The bound phage are recovered, and subsequently amplified by infecting E. coli. Monoclonal antibody-producing phage can be cloned by picking single plaques. In certain instances, the above process is repeated one or more times.

The antigen can be immobilized on a solid support to allow purification of antigen-binding phage by affinity chromatography. Alternatively, the antigen can be biotinylated, thereby allowing the separation of bound phage from unbound phage using streptavidin-coated magnetic beads. The antigen can also be immobilized on cells (for direct panning), in tissue cryosections, or on membranes (e.g., nylon or nitrocellulose membranes). Other variations of these panning procedures may be routinely determined by one skilled in the art.

Yeast display systems can also be used to produce monoclonal antibodies. In these systems, an antibody is expressed as a fusion protein with all or a portion of a yeast protein, for example the yeast AGA2 protein, which becomes displayed on the surface of the yeast cell wall. Yeast cells expressing antibodies with the desired binding selectivity can then be identified by exposing the cells to fluorescently labeled antigen, and isolated by flow cytometry (see, e.g., Boder and Wittrup, Nat. Biotechnol. 15:553-557, 1997).

Modified Cytochrome p450 or Ubiquitin Antibodies

Antibodies for use in immunoassays used to determine cytochrome p450 or ubiquitin levels in the bodily fluids of those at risk for breast cancer may include antibodies that are modified to alter one or more of the properties of the antibody. For some applications, a modified antibody may possess certain advantages over an unmodified antibody, such as increased affinity, for example. An antibody can be modified by linking it to a nonproteinaceous moiety, or by altering the glycosylation state of the antibody, e.g., by altering the number, type, linkage, and/or position of carbohydrate chains on the antibody, or altered so that it is not glycosylated.

In other modification techniques, one or more chemical moieties can be linked to the amino acid backbone and/or carbohydrate residues of the antibody. Certain exemplary methods for linking a chemical moiety to an antibody include, but are not limited to, acylation reactions or alkylation reactions (see, e.g., Malik et al., Exp. Hematol. 20:1028-1035, 1992, Francis, in “Focus on Growth Factors”, Vol. 3, No. 2, pp. 4-10 (Mediscript, Ltd., London, United Kingdom, 1992), European Patent Application Publication Nos. EP 0 401 384 and EP 0 154 316, and PCT Patent Application Publication Nos. WO 92/16221, WO 95/34326, WO 95/13312, WO 96/11953, and WO 96/19459). These reactions can be used to generate an antibody that is chemically modified at its amino-terminus for use in certain embodiments.

An antibody can also be modified by linkage to a detectable label, such as an enzymatic, fluorescent, isotopic or affinity label. Such a detectable label can allow for the detection or isolation of the antibody, and/or the detection of an antigen bound by the antibody in various immunoassays. Depending on the nature of the label, qualitative and/or quantitative determinations of cytochrome p450 or ubiquitin levels can be made using a colorimeter, a spectrophotometer, an ELISA reader, a fluorometer, or a gamma or scintillation (alpha or beta) counter that detects radioactive decay in assays utilizing isotope labels.

Affinity Maturation of Cytochrome p450 or Ubiquitin Antibodies

Higher affinity cytochrome p450 or ubiquitin antibodies are employed to provide significant advantages in certain embodiments of the cytochrome p450 or ubiquitin immunoassays described herein. Potential advantages include, but are not limited to, greater assay sensitivity, increased linearity, and decreased cost of goods. Antibody affinity may, in some cases, determine the formats that are available for use in an cytochrome p450 or ubiquitin immunoassays.

The affinity of an antibody for a particular antigen can be increased by subjecting the antibody to affinity maturation (or “directed evolution”) in vitro. In vivo, native antibodies undergo affinity maturation through somatic hypermutation followed by selection. Certain in vitro methods mimic that in vivo process, thereby allowing the production of antibodies having affinities that equal or surpass that of native antibodies.

In certain types of affinity maturation, mutations are introduced into a nucleic acid sequence encoding the variable region of an antibody having the desired binding selectivity (see, e.g., Hudson and Souriau, supra, and Brekke and Sandlie, Nat. Rev. Drug Discov. 2:52-62, 2002). Such mutations can be introduced into the variable region of the heavy chain, light chain, or both, into one or more CDRs, into H3, L3, or both, and/or into one or more FRs. A library of mutations can be created, for example, in a phage, ribosome, or yeast display library, so antibodies with increased affinity may be identified by standard screening methods (see, e.g., Boder et al., Proc. Natl. Acad. Sci. USA 97:10701-10705, 2000, Foote and Eisen, Proc. Natl. Acad. Sci. USA 97:10679-10681, 2000, Hoogenboom, supra, and Hanes et al., Proc. Natl. Acad. Sci. USA 95:14130-14135, 1998).

Mutations can be introduced by site-specific mutagenesis, based on information on the structure of the antibody, e.g., the antigen binding site, or using combinatorial mutagenesis of CDRs. Alternatively, all or a portion of the variable region coding sequence can be randomly mutagenized, e.g., using E. coli mutator cells, homologous gene rearrangement, or error prone PCR. Mutations can also be introduced using “DNA shuffling” (see, e.g., Crameri et al., Nature Med. 2:100-102, 1996, and Fermer et al., Tumour Biol. 25:7-13, 2004).

In addition, “chain shuffling” can be used to generate antibodies with increased affinity. In chain shuffling, one of the chains, e.g., the light chain, is replaced with a repertoire of light chains, while the other chain, e.g., the heavy chain, is unchanged, thus providing selectivity. A library of chain shuffled antibodies can be created, wherein the unchanged heavy chain is expressed in combination with each light chain from the repertoire of light chains. Such libraries may then be screened for antibodies with increased affinity. In particular applications, both the heavy and light chains are sequentially replaced, only the variable regions of the heavy and/or light chains are replaced, or only a portion of the variable regions, e.g., CDRs, of the heavy and/or light chains are replaced (see, e.g., Hudson and Souriau, supra, Brekke and Sandlie, supra, Kang et al., Proc. Natl. Acad. Sci. USA 88:11120-11123, 1991, and Marks et al., Biotechnology (NY) 10:779-783, 1992).

Mouse monoclonal antibodies that selectively bind human cytochrome p450 or ubiquitin or cytochrome p450 or ubiquitin from other mammals are subject to sequential chain shuffling. Such monoclonal antibodies include but not limited to, mouse monoclonal antibodies raised against mouse cytochrome p450 or ubiquitin but selectively bind to (i.e., cross-react with) human cytochrome p450 or ubiquitin. For example, the heavy chain of a given mouse monoclonal antibody can be combined with a new repertoire of human light chains, and antibodies with the desired affinity can be selected. The light chains of the selected antibodies can then be combined with a new repertoire of human heavy chains, and antibodies with the desired affinity can be selected. In this manner, human antibodies having the desired antigen binding selectivity and affinity can be obtained.

Alternatively, the heavy chain of a given mouse monoclonal antibody can be combined with a new repertoire of human light chains, and antibodies with the desired affinity selected from this first round of shuffling. In addition, the light chain of the original mouse monoclonal antibody is combined with a new repertoire of human heavy chains, and antibodies with the desired affinity selected from this second round of shuffling. Then, human light chains from the antibodies selected in the first round of shuffling are combined with human heavy chains from the antibodies selected in the second round of shuffling. Thus, human antibodies having the desired antigen binding selectivity and affinity can be selected.

Alternatively, a “ribosome display” method can be used that alternates antibody selection with affinity maturation. In the ribosome display method, antibody-encoding nucleic acid is amplified by RT-PCR between the selection steps. Thus, error prone polymerases may be used to introduce mutations into the nucleic acid (see, e.g., Hanes et al., supra).

Cytochrome p450 or Ubiquitin Antibody Binding Assays

Antibodies for use in various embodiments of the cytochrome p450 or ubiquitin immunoassays disclosed herein may be screened for binding to cytochrome p450 or ubiquitin (for example, human, mouse, dog, cat, horse, etc.) using routine methods that detect binding of an antibody to an antigen. In some embodiments, similar methods and assay formats are used to detect, and in some cases quantify, cytochrome p450 or ubiquitin levels in saliva obtained from patients that are thought to be at some risk for breast cancer (e.g., having DCIS breast cancer or at increased risk of developing DCIS breast cancer). Although the various protein markers described herein may be detected and quantified using any suitable technique that is known for detecting and quantifying antibodies, some exemplary techniques are summarized in the following discussion. For example, the ability of a monoclonal antibody to bind cytochrome p450 or ubiquitin may be assayed by standard immunoblotting methods, such as electrophoresis and Western blotting (see, e.g., “Current Protocols in Molecular Biology,” supra, Ch. 10.8, and “Antibodies: A Laboratory Manual”, supra). Alternatively, the ability of a monoclonal antibody to bind cytochrome p450 or ubiquitin may be assayed using a competitive binding assay, which evaluates the ability of a candidate antibody to compete with a known anti-cytochrome p450 or ubiquitin antibody for binding to cytochrome p450 or ubiquitin. Competitive binding assays may be performed in various formats including but not limited to ELISA (see, e.g., “Antibodies: A Laboratory Manual”, supra, Ch. 14) the results of which are determined using a colorimeter with one or more fixed wavelengths, or a variable wavelength spectrophotometer, or an ELISA reader, or a fluorometer. In some embodiments, such assays are used to determine cytochrome p450 or ubiquitin levels in bodily fluids obtained from patients that are thought to be at some risk for breast cancer.

A binding assay may be used to quantify the binding kinetics (e.g., rate constant) or the binding affinity (e.g., association or dissociation constant) of an antibody against cytochrome p450 or ubiquitin. The binding kinetics or binding affinity may be determined in the “solid-phase” by immobilizing antigen (e.g., cytochrome p450 or ubiquitin) on a solid support. In such assays, the immobilized antigen “captures” antibody from solution. Alternatively, binding kinetics or binding affinity may be determined using ELISA-based methods, or using biosensor-based technology, such as Biacore™ surface plasmon resonance technology (Biacore International AB, Uppsala, Sweden). Many such methods are known to those skilled in the art (see, e.g., “Antibody Engineering: A Practical Approach” (McCafferty et al., eds., Oxford University Press, Oxford, United Kingdom, 1996), Goldberg et al., Curr. Opin. Immunol. 5:278-281, 1993, Karlsson et al., J. Immunol. Methods 145:229-240, 1991, Malmqvist, Curr. Opin. Immunol. 5:282-286, 1993, and Hoogenboom, supra).

The binding kinetics or binding affinity of a Fab fragment that selectively binds to cytochrome p450 or ubiquitin may also be determined. Fab fragments do not multimerize. Multimerization may, in certain instances, complicate the measurement of binding kinetics and binding affinity in “solid phase” methods. Thus, Fab fragments that selectively bind to cytochrome p450 or ubiquitin may be suitable for use in certain binding assays in which antigen is immobilized to a solid support, such as, for example, an ELISA-based or Biacore™ assay. Fab fragments may be generated from an intact antibody that selectively binds to cytochrome p450 or ubiquitin using enzymatic methods, or by expressing nucleic acids encoding Fab fragments in a recombinant expression system.

Alternatively, the binding kinetics or binding affinity of an antibody against cytochrome p450 or ubiquitin may be determined using “solution phase” methods. The measurement of the binding kinetics or the binding affinity of multivalent antibodies and antibodies that multimerize are amenable to solution phase analysis. In such techniques, the kinetics or affinity of binding is measured for an antibody-antigen complex in solution. Such techniques are known to those skilled in the art, including, but not limited to, the “kinetic exclusion assay” (see, e.g., Blake et al., J. Biol. Chem. 271:27677-27685, 1996, and Drake et al., Anal. Biochem. 328:35-43, 2004). Sapidyne Instruments, Incorporated (Boise, Id.), among others, provides instrumentation for performing kinetic exclusion assays. These types of assays may be used to determine cytochrome p450 or ubiquitin levels in bodily fluids (e.g., saliva) obtained from patients that are thought to be at some risk for breast cancer in some instances.

Monoclonal antibodies raised for example against mouse cytochrome p450 or ubiquitin may be screened for selective binding to human, dog, cat or horse cytochrome p450 or ubiquitin using routine detection methods, such as those described herein. The ability of a monoclonal antibody to selectively bind both mouse and human cytochrome p450 or ubiquitin or those of other mammals (i.e., “cross-reactivity”) indicates the presence of the same epitope in mouse and human cytochrome p450 or ubiquitin or other mammal cytochrome p450 or ubiquitin. In detection methods that use denaturing conditions (e.g., Western blot), cross-reactivity indicates the monoclonal antibody binds to the same “linear” epitope in mouse and human cytochrome p450 or ubiquitin. In detection methods that use non-denaturing conditions, cross-reactivity indicates the monoclonal antibody binds to the same linear epitope or conformational epitope in mouse and human and other mammal cytochrome p450 or ubiquitin.

The epitope to which a monoclonal antibody binds may be identified by any of a number of assays (see, e.g., Morris, Methods Mol. Biol. 66:1-9, 1996). For example, epitope mapping may be achieved by gene fragment expression assays or peptide-based assays. In a gene fragment expression assay, for example, nucleic acids encoding fragments of cytochrome p450 or ubiquitin are expressed in prokaryotic cells and isolated. The ability of a monoclonal antibody to bind those fragments is assessed, e.g., by immunoblotting or immunoprecipitation. Nucleic acids encoding fragments of cytochrome p450 or ubiquitin can be transcribed and translated in vitro in the presence of radioactive amino acids. The radioactively labeled fragments of cytochrome p450 or ubiquitin can then tested for binding to a monoclonal antibody. Fragments of cytochrome p450 or ubiquitin can also be generated by proteolytic fragmentation. An epitope can also be identified using libraries of random peptides displayed on the surface of phage or yeast, or a library of overlapping synthetic peptide fragments of cytochrome p450 or ubiquitin, and testing for binding to a monoclonal antibody. An epitope can also be identified using a competition assay, such as those described below.

Monoclonal antibodies that bind to the same epitope of cytochrome p450 or ubiquitin as a monoclonal antibody of interest can be identified by epitope mapping, as described above, or by routine competition assays (see, e.g., “Antibodies: A Laboratory Manual”, supra, Ch. 14, and Morris, supra). In an exemplary competition assay, cytochrome p450 or ubiquitin, or a fragment thereof is immobilized onto the wells of a multi-well plate. The monoclonal antibody of interest is labeled with a fluorescent label (for example, fluorescein isothiocyanate) by standard methods, and then mixtures of the labeled monoclonal antibody of interest and an unlabeled test monoclonal antibody are added to the wells. The fluorescence in each well is quantified to determine the extent to which the unlabeled test monoclonal antibody blocks the binding of the labeled monoclonal antibody of interest. Monoclonal antibodies can be deemed to share an epitope if each blocks the binding of the other by 50% or more.

Alternatively, to determine if two or more monoclonal antibodies bind the same epitope, epitope binning may be performed (see, e.g., Jia et al., J. Immunol. Methods 288:91-98, 2004), using, for example, Luminex® 100 multiplex technology and the Luminex® 100™ analyzer (Luminex Corporation, Austin, Tex.). Epitope binning typically utilizes an antibody sandwich-type competition assay, in which a “probe” antibody is tested for binding to an antigen bound by a “reference” antibody. If the probe antibody binds to the same epitope as the reference antibody, it will not bind efficiently to the antigen, because that epitope is masked by the reference antibody. Immunoassays based on the above described technologies and devices (both those named and implied) are employed in various embodiments to detect cytochrome p450 or ubiquitin levels in the bodily fluids from patients that are thought to be at risk for breast cancer.

Antibodies directed against cytochrome p450 or ubiquitin, or conserved variants or peptide fragments thereof, which are discussed above, may also be used in identifying patients at high risk for breast cancer in diagnostic and/or prognostic assays, as described herein. Such diagnostic and/or prognostic methods may be used to detect abnormalities in the level of cytochrome p450 or ubiquitin in a patient's bodily fluid or tissues and may be performed in vivo or in vitro, such as, for example, on biopsy tissue. For example, antibodies directed to epitopes of cytochrome p450 or ubiquitin can be used in vivo to detect the level of cytochrome p450 or ubiquitin in the body. Such antibodies may be labeled, e.g., with a radio-opaque or other appropriate compound, and injected into a subject, in order to visualize binding to cytochrome p450 or ubiquitin expressed in the body, using methods such as X-rays, CAT-scans, or MRI.

Alternatively, immunoassays or fusion protein detection assays can be utilized on biopsy and/or autopsy samples in vitro to permit assessment of the expression pattern of cytochrome p450 or ubiquitin. Such assays can include the use of antibodies directed to epitopes of any of the domains of cytochrome p450 or ubiquitin. For example, in various embodiments antibodies, or fragments thereof, are used to quantitatively or qualitatively detect cytochrome p450 or ubiquitin, conserved variants, or peptide fragments thereof. This can be accomplished, for example, by immunofluorescence techniques employing a fluorescently labeled antibody coupled with ultraviolet microscopic, flow cytometric, or fluorometric detection.

The cytochrome p450 or ubiquitin antibodies (or fragments thereof), or cytochrome p450 or ubiquitin fusion or conjugated proteins, determining cytochrome p450 or ubiquitin levels may, additionally, be employed histologically, for example in immunofluorescence, immunoelectron microscopy, or non-immuno assays, for in situ detection of cytochrome p450 or ubiquitin or conserved variants or peptide fragments thereof. In situ detection may be accomplished by removing a histological specimen from a patient, and applying thereto a labeled antibody or fusion protein, performing some embodiments of a cytochrome p450 or ubiquitin immunoassay. The antibody (or fragment) or fusion protein is preferably applied by overlaying the labeled antibody (or fragment) onto a biological sample. Through the use of such a procedure, it is possible to determine not only the presence of cytochrome p450 or ubiquitin, or conserved variants or peptide fragments, but also its distribution in the examined tissue.

Immunoassays and non-immunoassays for cytochrome p450 or ubiquitin, or conserved variants or peptide fragments thereof, will typically comprise incubating a sample, such as a biological fluid, a tissue extract, freshly harvested cells, or lysates of cells that have been incubated in cell culture, in the presence of a detectably labeled antibody capable of identifying cytochrome p450 or ubiquitin, or conserved variants or peptide fragments thereof, and detecting the bound antibody by any of a number of techniques well-known in the art. The biological sample may be brought in contact with and immobilized onto a solid phase support or carrier such as nitrocellulose, or other solid support that is capable of immobilizing cells, cell particles, or soluble proteins. The support may then be washed with suitable buffers, followed by treatment with the detectably labeled cytochrome p450 or ubiquitin antibody or fusion protein. The solid phase support may then be washed with the buffer a second time to remove unbound antibody or fusion protein. The amount of bound label on solid support may then be detected by conventional means.

The terms “solid phase support” or “carrier” are intended to include any support or carrier capable of binding an antigen or an antibody. Well-known supports or carriers include, but are not limited to, glass, polystyrene, polypropylene, polyethylene, polyvinylidene fluoride, dextran, nylon, amylases, natural and modified celluloses, polyacrylamides, gabbros, and magnetite. The nature of the carrier may be either soluble to some extent or insoluble. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat, such as a sheet or test strip. Preferred supports include polystyrene beads. Those skilled in the art will know many other suitable carriers for binding antibody or antigen, or will be able to ascertain the same by use of routine experimentation.

The binding activity of a given lot of cytochrome p450 or ubiquitin antibody or cytochrome p450 or ubiquitin fusion protein may be determined according to well-known methods. Those skilled in the art will be able to determine operative and optimal assay conditions for each determination by employing routine experimentation.

With respect to antibodies, one of the ways in which a cytochrome p450 or ubiquitin antibody may be detectably labeled is by linking the same to an enzyme for use in an enzyme immunoassay (see, e.g., “Immunoassays: A Practical Approach” (Gosling, ed., Oxford University Press, Oxford, United Kingdom, 2000)). The enzyme that is bound to the antibody will react with an appropriate substrate, preferably a chromogenic substrate, in such a manner as to produce a chemical moiety that can be detected, for example, by spectrophotometric, fluorimetric, or visual means. These assays are read and analyzed using chromatometers, spectrophotmeters and fluorometers, respectively. Enzymes that can be used to detectably label the antibody include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, glucose oxidase, asparaginase, beta-galactosidase, ribonuclease, urease, catalase, glucose-6-phosphate dehydrogenase, glucoamylase and acetylcholinesterase. The detection may be accomplished by colorimetric methods that employ a chromogenic substrate for the enzyme. The detection may also be accomplished using methods that employ a fluorogenic substrate in an enzyme-lined fluorescence (ELF) assay. Detection may also be accomplished by visual comparison of the extent of enzymatic reaction of a substrate in comparison with similarly prepared standards.

Additionally, detection may also be accomplished using any of a variety of other immunoassays. For example, by radioactively labeling cytochrome p450 or ubiquitin antibodies or antibody fragments, it is possible to detect cytochrome p450 or ubiquitin through the use of a radioimmunoassay (RIA). The radioactive isotope may be detected, for example, by using a gamma or scintillation counter, or by autoradiography. Such antibodies or fragments may also be labeled with a fluorescent compound. When a fluorescently labeled antibody is exposed to light of the proper wavelength, it may be detected due to fluorescence. Exemplary fluorescent labeling compounds include, but are not limited to, fluorescein isothiocyanate, rhodamine, phycoerythrin, phycocyanin, allophycocyanin, o-phthaldehyde, and fluorescamine. Such antibodies can also be detectably labeled using a fluorescence emitting metal, such as ¹⁵²Eu, or others of the lanthanide series. These metals can be attached to an antibody or fragment using such metal chelating groups as diethylenetriaminepentacetic acid (DTPA) or ethylenediaminetetraacetic acid (EDTA).

A cytochrome p450 or ubiquitin antibody, or a fragment thereof, also may be detectably labeled by coupling it to a chemiluminescent compound. The presence of the chemiluminescent-tagged antibody or fragment is detected by luminescence that arises during the course of a chemical reaction. Examples of useful chemiluminescent labeling compounds include, but are not limited to, luminol, isoluminol, theromatic acridinium ester, imidazole, acridinium salt and oxalate ester. Likewise, a bioluminescent compound may be used to label the cytochrome p450 or ubiquitin antibodies, in some cases. Bioluminescence is a type of chemiluminescence found in biological systems, in which a catalytic protein increases the efficiency of the chemiluminescent reaction. The presence of a bioluminescent antibody or fragment is once again detected by luminescence. Exemplary bioluminescent compounds for purposes of labeling include, but are not limited to, luciferin, luciferase and aequorin (green fluorescent protein; see, e.g., U.S. Pat. Nos. 5,491,084, 5,625,048, 5,777,079, 5,795,737, 5,804,387, 5,874,304, 5,968,750, 5,976,796, 6,020,192, 6,027,881, 6,054,321, 6,096,865, 6,146,826, 6,172,188 and 6,265,548).

Immunochromatographic assays, also called lateral flow tests or simply strip tests, are a logical extension of the technology used in latex agglutination tests, the first of which was developed in 1956 by Singer and Plotz (Singer J. M. and Plotz C. M. The latex fixation test. I. Application to the serologic diagnosis of rheumatoid arthritis. Am. J. Med. 21, 888, 1956). The benefits of immunochromatographic tests include: their user-friendly format, short time to get test result, long-term stability over a wide range of climates and they are relatively inexpensive to make. These features make strip tests ideal for applications such as home testing, rapid point of care testing, and testing in the field, in places such as but not limited to the battlefield. In addition, they provide reliable testing that might not otherwise be available to rural environments or third world countries. Thus, such a format would have particular applicability in some cases by facilitating assessment of cytochrome p450 or ubiquitin levels at home or at a dentist's, physician's or veterinarian's office or other medical facility.

The principle behind the test is straightforward, any ligand that can be bound to a visually detectable solid support, such as dyed microspheres, may be tested for qualitatively, and in many cases even semi-quantitatively. Some of the more common lateral flow tests currently on the market are tests for pregnancy, Strep throat, and Chlamydia. The two predominant approaches to lateral flow tests are the non-competitive (or direct) and competitive (or competitive inhibition) reaction format. The non-competitive (or direct) double antibody sandwich reaction format is used when testing for larger analytes with multiple antigenic sites, such as, for example, LH, hCG, and HIV. In this format, less than an excess of sample analyte is desired, so that some of the microspheres will not be captured at the capture line, and will continue to flow toward the second line of immobilized antibodies, the control line. This is usually a species-specific anti-immunoglobulin antibodies, specific for the conjugate antibodies on the microspheres. The competitive reaction format is used most often when testing for small molecules with single antigenic determinants, which cannot bind to two antibodies simultaneously. If this format is chosen, it is important to pay close attention to the amount of antibody bound to the microspheres, in relation to the amount of free antigen in the sample. If the sample does not contain an excess of free antigen, some of the microspheres will bind at the capture line, giving a weak signal, and making the test result ambiguous. Typically, the membranes used to hold the antibodies in place are made up of primarily hydrophobic materials. Both the microspheres used as the solid phase supports and the conjugate antibodies are hydrophobic, and their interaction with the membrane allows them to be effectively dried onto the membrane.

One variation of the above reaction formats avoids the problem of protein-coated microspheres sticking to the membrane non-specifically by using a membrane that is inert and does not bind antibodies. This makes migration of the mobile phase antibodies very efficient and reliable. The capture antibodies, rather than being physically bound by the membrane, are attached to large microspheres, which will be held in place physically, rather than chemically, as the sample passes by, much like boulders in a stream. This may be used for both of the above-mentioned reaction formats. These principles are well-documented in the literature (See for example, U.S. Pat. Nos. 5,141,850, 5,160,701, 5,415,994, 5,451,504, 5,559,041, 5,886,154, 5,925,344, 6,093,804, 6,307,028, 6,913,888, 6,955,917, 7,358,055, 7,361,473, 7,427,490; published PCT patent application nos: WO 1988/08534 and WO 1991/12336; and published European patent Application Nos.: 0 284 232 A1 and 0 505 636 A1).

The technology involved in these lateral flow assays, provide an accurate, easy to use, rapid diagnostic tool. Additional approaches include using the same format for lateral flow tests and dyeing the solid support with a fluorescent dye, the possibility exists to create a truly quantitative test. If the spectral properties of the dyed microspheres to which the antibodies are conjugated are known, the amount of antibody bound at the capture line can be precisely quantified using a fluorometer. The benefit to this is that these tests could become truly quantitative assay. In addition, by placing multiple lines of capture antibodies on the membrane, each for a different analyte, a single test for more than one analyte may be developed. As an example, this format has been used to create a drugs-of-abuse test panel and Biosite's “Triage” was based also on this format (Bangs L. B. (1997) Immunological Applications of Microspheres. The Latex Course Biosite Company, 11030 Roselle St., San Diego, Calif. 92121).

Lateral flow assay technology is also used in the environmental field, where the format provides a rapid, reliable test that can be performed in the field for anything from water pollution to plant disease. Because these diagnostic tests are often performed in harsh environments, the lateral flow format is ideal. With proper preparation and foil pouching, no refrigeration or special handling is required. It also has use in the field of molecular genetics as a simple format for detecting various genetic markers, and DNA- or RNA-related infectious disease pathogens. The guiding principle behind this type of test, the ability to bind a ligand from solution to a solid support, can be performed on genetic material as well as proteins. Another method that can result in reduced development time is the use of protein-coated microspheres, such as ProActive® Streptavidin coated microspheres. By biotinylating the desired conjugate antibodies and then taking advantage of the strong affinity that biotin has for streptavidin, the antibodies are easily attached to the microspheres. Alternatively, Protein A or Protein G coated microspheres will bind many IgG's at the Fe region, allowing for optimized, directed antibody attachment. In this way, a series of assays can be developed rather quickly, using the same solid support, membrane, housing, etc. The only variable would be the conjugate and capture line antibodies used for each specific test. By combining several of these approaches a low-cost, rapid quantitative diagnostic assay for multiple analytes may be prepared for use in the field. For examples of use and methods of preparing such assays see, inter alia, U.S. Pat. Nos. 4,435,504, 4,594,327, 4,624,929, 4,756,828, 4,757,004, 4,837,395, 4,857,455, 4,911,794, 4,945,205, 4,956,275, 4,999,285, 5,039,607, 5,075,078, 5,087,556, 5,089,383, 5,164,294, 5,234,813, 5,248,619, 5,252,293, 5,998,221, 5,939,331, 5,908,757, 5,624,809, 5,529,752, 5,468,647, 5,451,507, 5,435,970, 5,334,513, 6,194,220, 6,277,646, 6,686,167, 6,855,561, 6,969,591, 7,067,264, 7,179,657 and 7,226,793; and published U.S. patent application nos.: 20080160549, 20070281370, 20070190531, 20070111323, 20060160084, 20040068101, 20040058395, 20030211634, 20030157699, 20030100128, 20030049167, 20030027222, 20020106696, 20020085958.

An immunochromatographic assay, or lateral flow test for the level of cytochrome p450 or ubiquitin in a sample of a patients bodily fluid, such as saliva, would be very useful in some embodiments of the testing methods described herein, as it provides several potential benefits. For example, in some applications, it may be used in a kit designed to facilitate the process of identifying individuals at increased risk for breast cancer in rural areas or even in a physician, dentist or veterinarian's office or public clinic. In some embodiments such an assay need be only semi-quantitative as only those samples whose cytochrome p450 are decreased or ubiquitin levels are increased above the normal range need be detected. As an immunoassay, this test could be titrated to obtain the desired cutoff and sensitivity.

Nephelometry is a technique performed by shining light on a sample, and measuring the amount of light scattered. This technique is widely used in clinical laboratories because it is relatively easily automated. It is based on the principle that a dilute suspension of small particles will scatter light (often but not necessarily a laser) passed through it rather than simply absorbing it. The amount of scatter is determined by collecting the light at an angle (usually at about 70 or 75 degrees). Such immunoturbidimetric assays (protein immunoassays) are read using a Photometer (such as a DTN-410, DIALAB GmbH, Germany) or autolyzer Photometer (DTN-410K, DIALAB GmbH). Antibody and the antigen are mixed in concentrations such that only small aggregates are formed that do not quickly settle to the bottom. The amount of light scatter is measured and compared to the amount of scatter from known mixtures. The amount of the unknown is determined from a standard curve.

Enzyme multiplied immunoassay technique, or EMIT, is a common method for screening urine and blood for drugs, both legal and illicit. First introduced by Syva Company in 1973, it is the first homogeneous immunoassay to be widely used commercially. A mix and read protocol has been developed that is exceptionally simple and rapid. The most widely used applications for EMIT are for therapeutic drug monitoring (serum) and as a primary screen for abused drugs and their metabolites (urine). Early patents covering the major aspects of the method are U.S. Pat. Nos. 3,817,837 and 3,875,011. While still sold by Siemens Healthcare under its original trade name, EMIT, assay kits with different names that employ the same technology are supplied by other companies. The method is highly reliable and reliance on its results has even been upheld by the US Supreme Court. Older ELISAs utilize chromogenic substrates, while many of the newer assays employ fluorogenic substrates enabling increased sensitivity, both types can be read on an ELISA microplate reader (such as the DIAREADER:DIALAB GmbH, Germany) of the appropriate measuring range, for example from 400 nm n to 750 nm.

Additionally, analytical scale immunoprecipitations can also be used to detect the presence of an analyte, such as cytochrome p450 or ubiquitin in bodily fluids, preferably saliva. Monodispersed magnetic beads are also available as a support material which offers certain advantages over polydisperse agarose beads. Magnetic beads have the ability to bind extremely large protein complexes and the complete lack of an upper size limit for such complexes, as unlike agarose beads which are sponge-like porous particles of variable size, magnetic beads are small, solid and (in the case of monodisperse magnetic beads) spherical and uniform in size. The lower overall binding capacity of magnetic beads for immunoprecipitation make it much easier to match the quantity of antibody needed for diagnostic immunoprecipitations precisely with the total available binding capacity on the beads which results in decreased background and fewer false positives. The increased reaction speed of the immunoprecipitations using magnetic bead technologies results in superior results when the analyte protein is labile due to the reduction in protocol times and sample handling requirements which reduces physical stresses on the samples and reduces the time that the sample is exposed to potentially damaging proteases. Agarose bead-based immuno-precipitations may also be performed more quickly using small spin columns to contain the agarose resin and quickly remove unbound sample or wash solution with a brief centrifugation (Celis, J. E., Lauridsen, J. B., and Basse, B. (1994) Determination of antibody specificity by Western blotting and immunoprecipitation. In: Celis, J. E. (ed.), CELL BIOLOGY. A LABORATORY HANDBOOK, Academic Press, New York, Vol. 2, pp. 305-313. Mason, D. W., and Williams, A. F. (1986) Kinetics of antibody reactions and the analysis of cell surface antigens. In: Weir, D. M., Herzenberg, L. A., Blackwell, C., and Herzenberg, L. A. (ed.), HANDBOOK OF EXPERIMENTAL IMMUNOLOGY, Blackwell, Oxford, vol. 1, chapter 38.

### EXAMPLES

### Example 1

### Salivary Protein Analyses With iTRAQ™

Saliva samples are thawed and immediately centrifuged to remove insoluble materials. The supernatant is assayed for protein using the Bio-Rad protein assay (Hercules, Calif., USA) and an aliquot containing 100 μg of each specimen is precipitated with 6 volumes of −20° C. acetone. The precipitate is resuspended and treated according to the iTRAQ™ manufacturer's instructions. Protein digestion and reaction with iTRAQ™ labels are carried out according to the manufacturer's instructions (Applied Biosystems, Foster City, Calif.). Briefly, the acetone precipitable protein is centrifuged in a table top centrifuge at 15,000×g for 20 minutes. The acetone supernatant is removed and the pellet resuspended in 20 μl dissolution buffer. The soluble fraction is denatured and disulfides reduced by incubation in the presence of 0.1% SDS and 5 mM TCEP (tris-(2-carboxyethyl)phosphine)) at 60° C. for one hour. Cysteine residues are blocked by incubation at room temperature for 10 minutes with MMTS (methyl methane-thiosulfonate). Trypsin is added to the mixture to a protein:trypsin ratio of 10:1. The mixture is incubated overnight at 37° C. The protein digests are labeled by mixing with the appropriate iTRAQ™ reagent and incubating at room temperature for one hour. On completion of the labeling reaction, the four separate iTRAQ™ reaction mixtures are combined. Since there are a number of components that might interfere with the LC-MS/MS analysis, preferably the labeled peptides are partially purified by a combination of strong cation exchange followed by reverse phase chromatography on preparative columns, employing techniques that are known in the art. The combined peptide mixture is diluted 10 fold with loading buffer (10 mM KH2PO4 in 25% acetonitrile at pH 3.0) and applied by syringe to an iCAT Cartridge-Cation Exchange column (Applied Biosystems, Foster City, Calif.) column that has been equilibrated with the same buffer. The column is washed with 1 ml loading buffer to remove contaminants. To improve the resolution of peptides during LCMSMS analysis, the peptide mixture is partially purified by elution from the cation exchange column in 3 fractions. Stepwise elution from the column is achieved with sequential 0.5 ml aliquots of 10 mM KH2PO4 at pH 3.0 in 25% acetonitrile containing 116 mM, 233 mM and 350 mM KCl respectively. The fractions are evaporated by Speed Vac to about 30% of their volume to remove the acetonitrile and then slowly applied to an Opti-Lynx Trap C18 100 μl reverse phase column (Alltech, Deerfield, Ill.) with a syringe. The column is washed with 1 ml of 2% acetonitrile in 0.1% formic acid and eluted in one fraction with 0.3 ml of 30% acetonitrile in 0.1% formic acid. The fractions are dried by lyophilization and resuspended in 10 μl 0.1% formic acid in 20% acetonitrile. Each of the three fractions is analyzed by reverse phase LCMSMS.

Reverse Phase LC-MS/MS.

The desalted and concentrated peptide mixtures are quantified and identified by nanoLC-MS/MS on an API QSTAR™ XL mass spectrometer (ABS Sciex Instruments) operating in positive ion mode. The chromatographic system consists of an UltiMate™ nano-HPLC and FAMOS autosampler (Dionex LC Packings). Peptides are loaded on a 75 μm×10 cm, 3 μm fused silica C18 capillary column, followed by mobile phase elution: buffer (A) 0.1% formic acid in 2% acetonitrile/98% Milli-Q™ water and buffer (B): 0.1% formic acid in 98% acetonitrile/2% Milli-Q™ water. The peptides are eluted from 2% buffer B to 30% buffer B over 180 minutes at a flow rate 220 mL/min. The LC eluent is directed to a NanoES source for ESI/MS/MS analysis. Using information-dependent acquisition, peptides are selected for collision induced dissociation (CID) by alternating between an MS (1 sec) survey scan and MS/MS (3 sec) scans. The mass spectrometer automatically chooses the top two ions for fragmentation with a 60 s dynamic exclusion time. The IDA collision energy parameters were optimized based upon the charge state and mass value of the precursor ions. For each saliva sample set there are three separate LC-MS/MS analyses.

The accumulated MSMS spectra are analyzed by ProQuant™ and ProGroup software packages (Applied Biosystems) using the SwissProt™ fasta database for protein identification. The ProQuant™ analysis is carried out with a 75% confidence cutoff with a mass deviation of 0.15 Da for the precursor and 0.1 Da for the fragment ions. The ProGroup reports are generated with a 95% confidence level for protein identification.

Bioinformatics.

The Swiss-Prot™ database is employed for protein identification while the PathwayStudio® bioinformatics software package is used to determine regulatory pathways and distribution of proteins according to function. Venn diagrams may be constructed, using, for example, the Venn Diagram Plotter software program presently available from the U.S. National Center for Research Resource. Graphic comparisons with log conversions and error bars for protein expression are produced using the ProQuant™ software, for example.

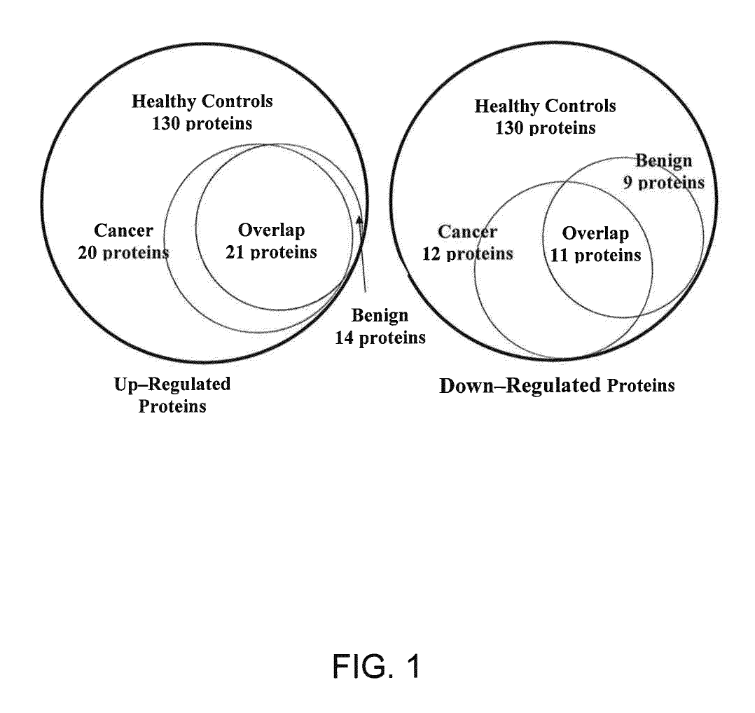

Table 1 summarizes the results of an exemplary iTRAQ™ analysis of saliva samples carried out as described above. Overall protein comparisons between benign vs. healthy, cancer vs. benign and cancer vs. healthy adult female subjects are shown. In total, 130 proteins were identified at a confidence level >95, and of those, 72 proteins were identified at >99 confidence level. Of these 130 proteins, there were 40 proteins that were determined to be expressed significantly differently (p<0.05) in the benign or tumor saliva compared to healthy controls. FIG. 1 is a Venn diagram of these proteins showing the overlapping proteins among the three groups of women.

Table 2 contains a list of the up-regulated (n===14) and down-regulated (n=9) proteins for the pooled saliva sample composed of individuals diagnosed with a fibroadenoma (benign tumor). The fold increase of protein and p-values are also presented. As shown in Table 2, of the 29 proteins, 9 (69%) were significant at the p<0.001 to p<0.0001 levels and 7 of those 9 proteins had a greater than 50% change in concentration.

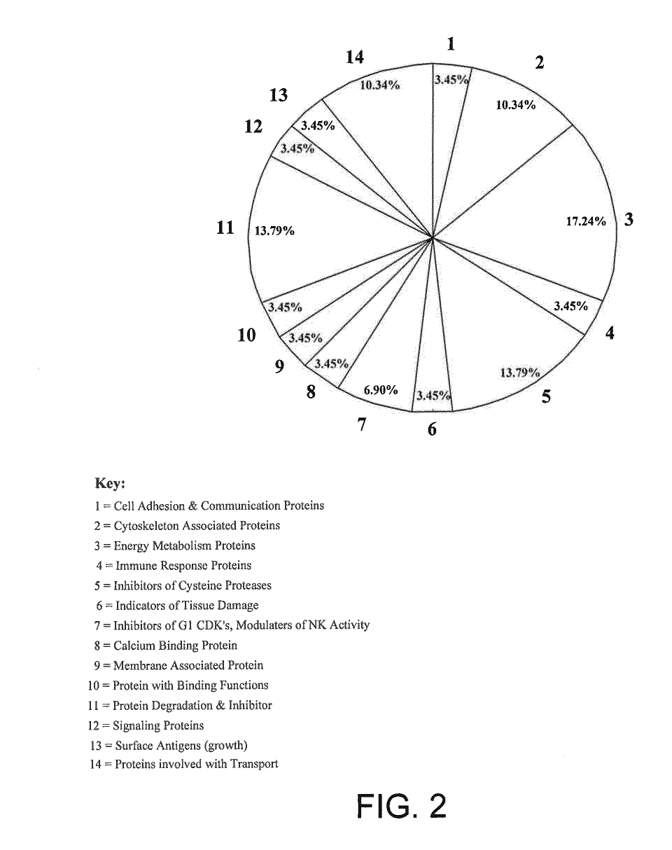

Table 3 contains a list of the up-regulated (n=20) and down-regulated (n=12) proteins observed in the Stage 0 cancer saliva compared to controls. “Stage 0 cancer” refers to breast tumor in which the stage is microscopic and demonstrates in situ involvement. The Stage 0 cancer saliva samples were obtained from women diagnosed with ductal carcinoma in situ. There were 15 proteins that showed a 1.5 fold increase in levels in the cancer compared to control subjects. Of these 15 differentially expressed proteins, 12 were significant at the p<0.001 to p<0.0001 levels. Each of the proteins listed in Table 3 is referenced in the literature as having been found in blood from cancer subjects and/or in cell supernatants from cancer cell lines. Of the 32 proteins that were up- or down-regulated secondary to carcinoma of the breast, 79% of those differentially expressed proteins are cited in the literature as being involved, molecularly, with breast cancer. The functions attributed to these proteins are shown in FIG. 2, along with an indication of the percentage of the total number of proteins that correspond to those respective functions.

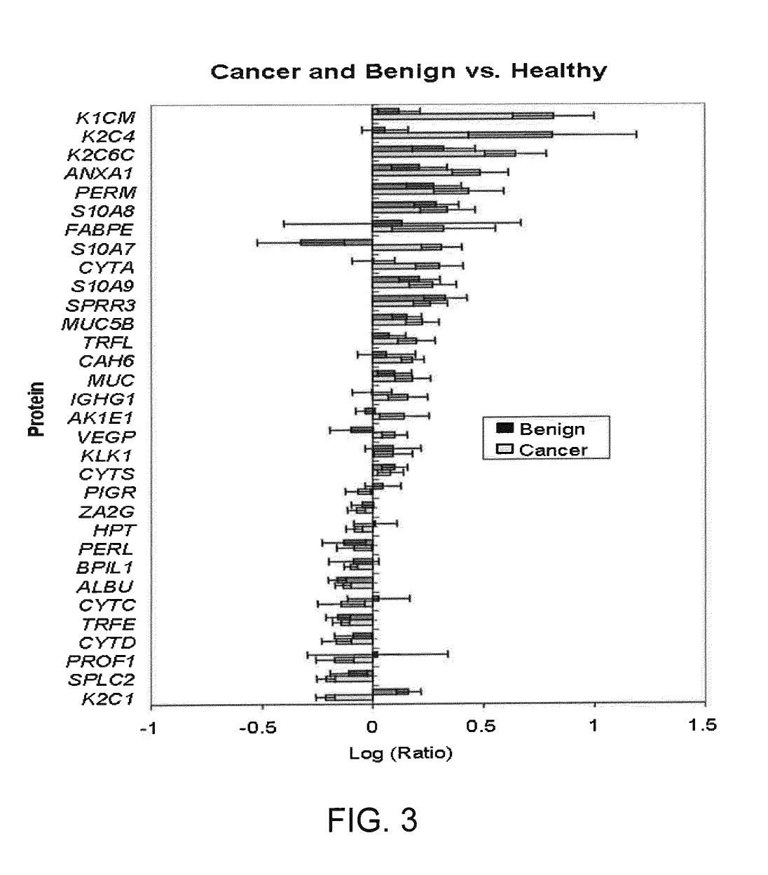

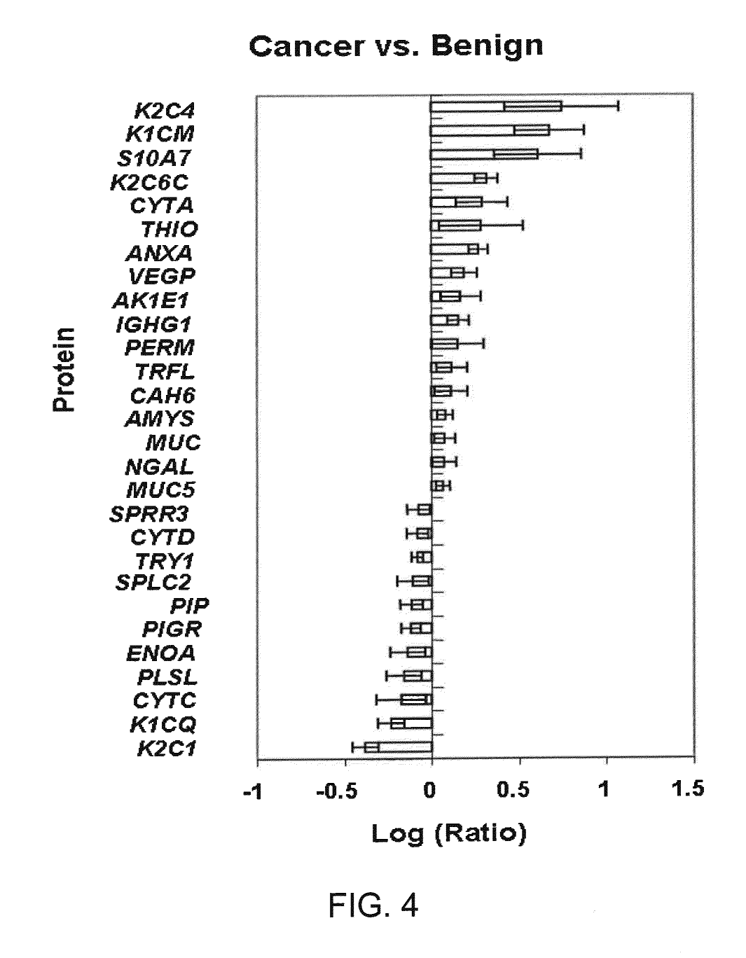

A comparison of the differentially expressed proteins is shown in graphical form in FIG. 3. In this figure the log of the ratios for benign vs. control and cancer vs. control is plotted for each of the proteins. The error bars are the log of the error factor calculated by the ProQuant™ software. This comparison illustrates that there are a number of significant differences in the expression levels of several proteins between the cancer and benign saliva samples. A direct comparison of protein expression ratios between the benign and cancer pooled specimens that exhibited overlap or commonality among the proteins is shown in Table 4. Among the comparison of the overlapping proteins, the levels of 10 proteins fell at or below a p-value of 0.001 and the levels of 9 proteins represented a greater than 50% difference in the cancer saliva samples compared to benign saliva samples. These data are replotted in log form in FIG. 4.

In brief, three pooled (n=10 subjects/pool) stimulated whole saliva specimens from women were analyzed. One pooled specimen was from healthy women, another pooled specimen from women diagnosed with a benign breast tumor and the other one pooled specimen was from women diagnosed with ductal carcinoma in situ (DCIS). Differential expression of proteins was measured by isotopically tagging proteins in the tumor groups and comparing them to the healthy control group. Experimentally, saliva from each of the pooled samples was trypsinized and the peptide digests labeled with the appropriate iTRAQ™ reagent. Labeled peptides from each of the digests were combined and analyzed by reverse phase (C18) capillary chromatography on an Applied Biosystems QStar™ LC-MS/MS mass spectrometer equipped with an LC-Packings HPLC.

With respect to the overall analyses, the total number of salivary proteins reported in healthy individuals at the 95% confidence levels was 130 in the exemplary study disclosed herein. By comparison, in prior studies that used 2D gel and mass spectrometry, about 100-102 cancer-related salivary proteins were reported (Wilmarth, et al. J Prot Res. 2004, 3, 1017-23; 24; Ghafouri, et al. Proteomics. 2003, 3(6), 1003-15). In still other prior studies, about 300 cancer-related salivary proteins were reported based on both 2D gel and “shotgun” proteomic techniques (Hu, et al. Proteomics. 2005, 5, 1714-28.) Differences in the number of total proteins identified are probably a result of different technologies, profiling based on single samples or in collection and/or using single individual profiling (non-pooled specimen) or in collection and/or sampling techniques (Streckfus, et al. Salivary biomarkers for the detection of cancer. In. Progress in Tumor Marker Research, Swenson L I ed. Nova Scientific Publishing, Inc., New York, 2007.)

Of the 130 proteins presently identified in the saliva specimens, forty nine proteins were differentially expressed between the healthy control pool and the benign and cancer patient groups. Table 3 lists the proteins for the healthy pool vs. benign tumor pool, and Table 4 lists the proteins for the healthy pool vs. cancer tumor pool. As illustrated in Table 4, many of these proteins have been reported as being either up- or down-regulated in blood and cancer tissue. There is also an overlap of 13 up-regulated proteins and five down-regulated proteins between the protein profiles, leaving the benign group with five proteins that are unique to fibroadenomas (ENOA, IGHA2, IL-Ira, S10A7, SPLC2) and 11 proteins unique to DCIS (CAH6, K2C4, CYTA, FABP4, IGHGI, TRFL, BPIL1, CYTC, HPT, PROF1, ZA2G). FIG. 1 shows Venn diagrams of the overlapping proteins between the three groups of women. Notably, two-thirds of the total “overlap” proteins were up-regulated. Without wishing to be limited to any specific theory to explain this phenomenon, it is proposed that a portion of the up-regulated proteins that exhibited overlap are associated with pathways which are common to both disorders. This would include the proteins associated with cytoskeleton and cell growth. The benign tumor was targeted in order to increase the specificity of the panel of markers. If there are markers specific to a benign tumor and other markers specific only to the malignancy, then the probability of making the correct clinical assessment is further increased.

Tables 5, 6 and 7 represent a comparison of the healthy control and cancer proteins which overlapped each group. As illustrated in Tables 5-7, changes are listed in order of confidence, level 3 being the highest confidence, Confidence levels are defined as follows: Level 3: Changes greater than 2 fold from good quality signals that also pass a visual inspection. Level 2: Changes greater than 2 fold from good quality signals that do not pass visual inspection. Level 1: Changes greater than 2 fold from low quality signals. Level 0: No significant protein changes.

In this comparison, only seven proteins remained significantly different in the presence of carcinoma (≦p<0.005). This would include the proteins associated with exocytosis, the cytoskeleton and immuno-response. As the cell proliferation process is further enhanced in the presence of carcinoma, these proteins are expected to be significantly up-regulated in the case of carcinoma. FIGS. 3 and 4 provide further illustration of the protein comparisons.

Without wishing to be limited to a single theory to explain the mechanism by which these proteins are altered in the presence of carcinoma of the breast, it is proposed that since the histo-physiology is very similar between the ductal tissues of the breast and those of the salivary glands, there may be extra-cellular communication between the two distant tissues (Wick, et al. Am J Clin Pathol. 1998, 109, 75-84; Kurachi, et al. Proc Natl Acad Sci USA. 1985, 82, 5940-5943). This phenomenon has also been observed in nipple aspirates (Kuerer, H. M. et al. Clin Canc Res. 2003, 9, 601-605; Alexander, et al. Clin Cancer Res. 2004, 10(22), 7500-10) which have yielded many of the same protein constituents as observed in Table 4.

The salivary proteome that is altered in the presence of carcinoma of the breast was examined in the present studies, and a group of proteins were identified that have potential diagnostic utility for breast cancer. Saliva as a diagnostic media has potential clinical advantages because it contains numerous proteins and protein fragments that may have analytical value. Salivary fluid is continually produced and excreted in an open-ended circuit, thereby offering a way to obtain “real-time” results. In contrast, blood exists in a “closed-loop” system. Therefore blood, as a circulating media, may contain proteins that are a day, a week, or a month old as well as proteins which have passed numerous times through many organ systems or have been excreted (Streckfus, et al. Exp Rev Prot. 2007, 4(3), 329-332). In some cases, saliva and nipple aspirates may be a more useful diagnostic fluid than blood.

### Example 2

### Use of Salivary Biomarkers to Differentiate Non-Cancerous Tissue, Benign Tumor, and DCIS in a Test Sample

In some embodiments, one or more salivary biomarkers are used to differentiate non-cancerous breast tissue, benign breast tumor and ductal carcinoma in situ of the breast by analyzing the salivary proteome of an individual suspected of having breast cancer, as described above. One or more of the protein biomarkers identified in Tables 3 and 4 are identified and quantified in the test patient's saliva specimen, and the resulting values are then compared to a biomarker reference panel, which is developed in accordance with the above-described procedure. The biomarker reference panel is made up of a group of the same saliva protein constituents developed using DCIS, benign tumor and healthy, non-cancerous (i.e., tumor free) control group populations. Each constituent has associated with it a range of concentration values, a mean concentration, and statistical error range. Receiver Operating Characteristic Curves (ROC) curves (sensitivity vs. 1-specificity) are constructed for salivary protein concentrations. The optimum cutoff value for each marker is determined by using the cutoff value that produces the largest percentage of area under its ROC curve. The salivary ROC curves for each marker are compared using a modified Wilcoxon rank sum procedure. Once determinations have been made for the sensitivity and specificity of each marker, positive and negative predictive values, and likelihood ratios are calculated. The same types of analyses are also made for the benign tumor group versus the cancer group. From comparison of the individual marker values to the reference panel values, a differential diagnosis of the patient is determined.

For individual protein biomarker values that fall within the range of control values, a diagnosis of lower risk or likelihood of occurrence of breast tumor is determined. A comparison chart similar to that shown in FIG. 3 or 4 may be employed, for example, in which the reference values of the biomarkers in breast tumor saliva samples are presented relative to controls (e.g., mean values for saliva samples from breast tumor-free individuals). Alternatively, the reference values may be stored electronically on a computer readable storage device, and a computer aided comparison is performed and a diagnostic report is prepared, upon input of the test sample biomarker levels. When the concentration value of a selected protein biomarker falls within the range for that protein in the DCIS and/or benign groups of the reference panel, a diagnosis of elevated risk or likelihood of the presence or occurrence of a breast tumor is appropriate. When the concentration of a protein is significantly different than the mean concentration of the same protein in the control group of the reference panel, a diagnosis of high risk or high likelihood of the presence of a breast tumor, or of the occurrence of a breast tumor, is appropriate.

### Example 3

In one embodiment of a screening procedure, the biomarker Q9UBC9/SPRR3 (encoded by GenBank Accession No. Q9UBC9 Gene ID. SPRR3), indicated in Tables 3 and 4, is quantitated in the saliva of an individual to diagnose a breast cancer, using the above-described saliva sample preparation and analysis procedures, or equivalent methods. The detected level or value of the biomarker is then compared to reference values of the biomarker in the saliva of individuals with breast tumors (either benign fibroadenomas or DCIS). By comparison of the individual's marker value to the reference value (or range of values), a differential diagnosis of the patient is determined, to differentiate between breast cancer and breast cancer-free condition.

In a modification of this procedure, the individual's salivary level of the Q9UBC9/SPRR3 biomarker is additionally compared to respective reference values in the saliva of individuals with DCIS and of individuals with benign breast tumor (i.e., fibroadenoma). By further comparison of the individual's marker value to the respective reference values of the marker in individuals with DCIS or benign breast tumor, a further diagnosis of the patient is obtained, to differentiate between benign breast tumor and DCIS.

### Example 4

In another embodiment, the biomarker Q8N4F0/BPIL1 (encoded by GenBank Accession No. Q8N4F0 Gene ID. BPIL1), indicated in Table 3, is quantitated in the saliva of an individual to diagnose DCIS breast tumor, using the above-described saliva sample preparation and analysis procedures, or equivalent methods. The detected level of the biomarker is then compared to respective reference values of the biomarker in the saliva of individuals with DCIS and of individuals with benign breast tumor (i.e., fibroadenoma). By comparison of the individual's marker value to the reference values, a differential diagnosis of the patient is determined, to differentiate between tumor-free breast tissue and a breast cancer, or to indicate lower risk or likelihood of the presence or occurrence, if the value of the test sample falls within the control values for this protein biomarker.

### Example 5

In another embodiment, the biomarker P07737/PROF1 (encoded by GenBank Accession No. P07737 Gene ID. PROF1), indicated in Table 3, is quantitated in the saliva of an individual to diagnose benign or DCIS breast tumor, using the above-described saliva sample preparation and analysis procedures, or equivalent methods. The detected level of the biomarker is then compared to respective reference values of the biomarker in the saliva of individuals with DCIS and of individuals with benign breast tumor (i.e., fibroadenoma). By comparison of the individual's marker value to the reference values, a differential diagnosis of the patient is determined, to differentiate between tumor-free breast tissue and a breast tumor (either benign or DCIS). As noted above, if the value of the test sample falls within the control values for this protein biomarker, a diagnosis of lower risk or likelihood of the presence or occurrence of either fibroadenoma or DCIS is made.

### Example 6

Another embodiment uses the biomarker P01024/C03 (encoded by GenBank Accession No. P01024 Gene ID. C03). No. P01024 Gene ID C03 is important due to its central role in the activation of the classical complement system and contributes to innate immunity. Its activation is required and generally results in an early inflammatory response. As DCIS is a very early stage carcinoma and localized inflammation may be initially present, this protein will present itself as a marker for early detection. This biomarker is quantitated in the saliva of an individual using the above-described saliva sample preparation and analysis procedures, or equivalent methods. The detected level of the biomarker is then compared to a reference value of the biomarker in the saliva of individuals with DCIS. By comparison of the individual's marker value to the reference value, a diagnosis of the patient is determined, to differentiate between tumor-free breast tissue and DCIS. As noted above, if the value of the test sample falls within the control values for this protein biomarker, a diagnosis of lower risk or likelihood of the presence or occurrence of DCIS is made. This biomarker is not typically associated with benign tumors. Therefore, in some screening applications, the comparative values of the P01024/C03 biomarker in an individual's saliva will be especially informative as to the likelihood of the risk or present existence of DCIS in that individual.

### Example 7

In variations of the foregoing embodiments, quantitation and analysis of two or more of the above-identified biomarkers are combined to increase the robustness of the diagnostic test. For example, a screening procedure may analyze the comparative levels of Q9UBC9/SPRR3 and one or more biomarkers that are essentially unique to fibroadenomas (e.g., ENOA, IGHA2, IL-1ra, S10A7, SPLC2). In some cases, a screening procedure may analyze the comparative levels of Q8N4F0/BPIL1 and one or more biomarkers that are essentially unique to DCIS (e.g., CAH6, K2C4, CYTA, FABP4, IGHGI, TRFL, BPIL1, CYTC, HPT, PROF1 and ZA2G), in an individual's saliva sample. In some cases, a screening procedure may analyze the comparative levels of P07737/PROF1 or P01024/C03 in an individual's saliva relative to the respective biomarker value in control saliva samples, to determine a diagnosis of the patient that differentiates between tumor-free breast tissue and breast tumor.

### Example 8

It is believed that the protein biomarkers Q9UBC9/SPRR3, Q8N4F0/BPIL1, P07737/PROF1 and P01024/C03 have not been previously associated with breast tumors. They are believed to primarily function, respectively, as an indicator of tissue damage, as a transport protein, i.e. associated with movement of proteins across a cellular membrane, as a cytoskeleton-associated protein, and as an initiator of immune responses. The combined screening for saliva levels of variously-functioning protein biomarkers such as these potentially offers a more robust diagnostic method than a method that screens for protein biomarkers associated with only a single physiological function. Additionally, using multiple biomarkers enhances cancer detection by reducing the number of false positives and negatives. This is achieved by using proteins that are associated uniquely with specific biological pathways, markers that are tumor specific (benign vs. malignant), and by determining if the proteins are up- or down-regulated in the presence of disease. Collectively, this information will reduce subjectivity and provide the clinician with information for superior clinical decision-making.

Accordingly, in another embodiment of a diagnostic screening procedure, one or more of the biomarkers Q9UBC9/SPRR3, Q8N4F0/BPIL1, P07737/PROF1 and P01024/C03 are quantitated and analyzed along with another biomarker, such as a biomarker that is known to be significantly up-regulated or down-regulated in benign breast tumors and/or DCIS (e.g., significant at the p<0.001 to p<0.0001 level). Such other biomarkers include, for example, those that are known to be associated with cell adhesion and/or communication; are associated with the cytoskeleton; are involved with energy metabolism; are associated with immune response; are inhibitors of cysteine proteases; are indicators of tissue damage; inhibit G1 CDKs, or modulate NK activity; are calcium binding proteins; are membrane associated proteins; are proteins with binding functions; are involved with protein degradation and inhibition; are associated with cell signaling; are surface antigens related to growth; or are involved with transport in cells of the body. Some specific examples of such biomarkers are identified in Tables 3 and 4.

### Example 9

### Monitoring Breast Cancer Patients after Surgery

A patient may be monitored for recurrence or progression of breast cancer after surgery, by testing the status of saliva biomarkers before and after surgery, and periodically thereafter. Such differential identifications may be used alone or in conjunction with one or more other diagnostic methods for diagnosing or monitoring a patient for breast cancer. For instance, the patient may have received therapeutic treatment for breast cancer, with or without prior surgical removal of cancerous tissue. Analyzing saliva samples for protein biomarkers, in accordance with the methods described herein, will potentially aid in making treatment decisions for the patient. The effectiveness of a given drug, or radiation therapy, or surgical procedure may be monitored by periodically determining the status of the saliva biomarkers in the patient, and comparing them to the same reference panel and/or to the patient's previous saliva tests.

The potential diagnostic benefits include the overall management of breast cancer in women. The diagnosis of breast cancer at an earlier stage allows a woman more choice in selection of various treatment options. A saliva based test is potentially useful in the postoperative management of cancer patients. For example, in some cases, following tumor removal, a decrease in marker concentration will follow and eventually plateau to within a normal level indicating that the patient is free of disease. In contrast, a persistently high level of salivary protein biomarkers will be indicative of tumor recurrence or persistence. Saliva is potentially a cost effective method for monitoring the effectiveness of chemotherapy in which decreases in marker concentrations are observed if the treatment regimen is effective.

### Example 10

### Isotopic Labeling and Liquid Chromatography Tandem Mass Spectroscopy to Identify Potential Salivary Protein Markers for Breast Cancer

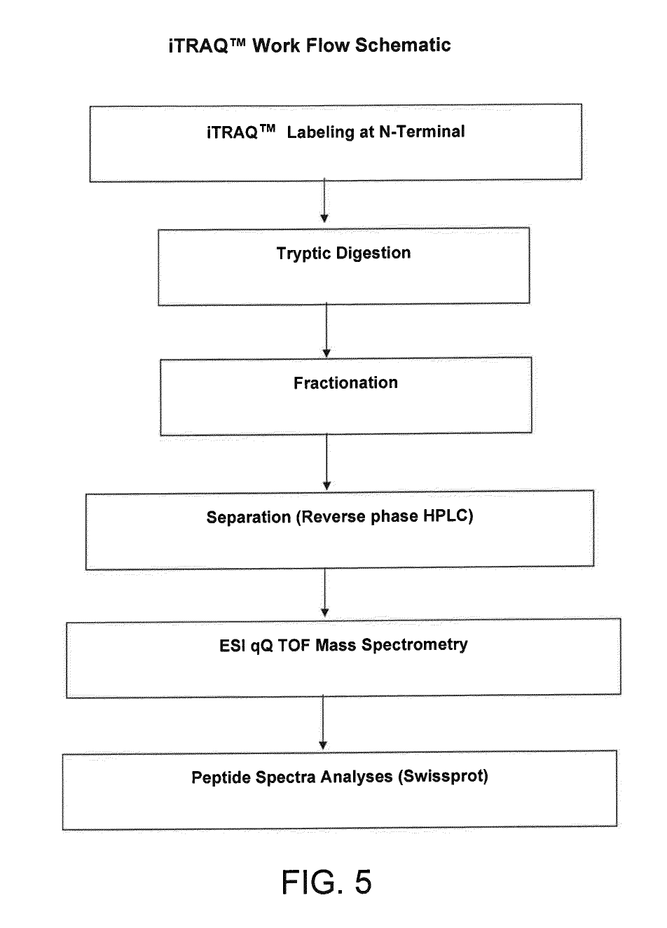

In further tests, the IL-LC tandem MS technique was used to protein profile saliva for novel cancer-related biomarkers. To identify potential salivary protein markers for the detection of breast cancer the following were used: pooled (N==10) saliva specimens from healthy subjects; pooled (N=10) saliva specimens from benign tumor patients (fibroadenomas); pooled (N=10) saliva specimens from stage 0 cancer subjects; and pooled (N=10) saliva specimens from stage I breast cancer subjects. An internal standard was created by pooling 10 specimens randomly selected from the pooled population. The analytical matrix is shown in the iTRAQ™ work flow schematic of FIG. 5.

The saliva samples were thawed and immediately centrifuged to remove insoluble materials. The supernatant was assayed for protein (Bio-Rad, San Diego, Calif.) and an aliquot containing 100 mg of each specimen was precipitated with six volumes of −20° C. acetone.

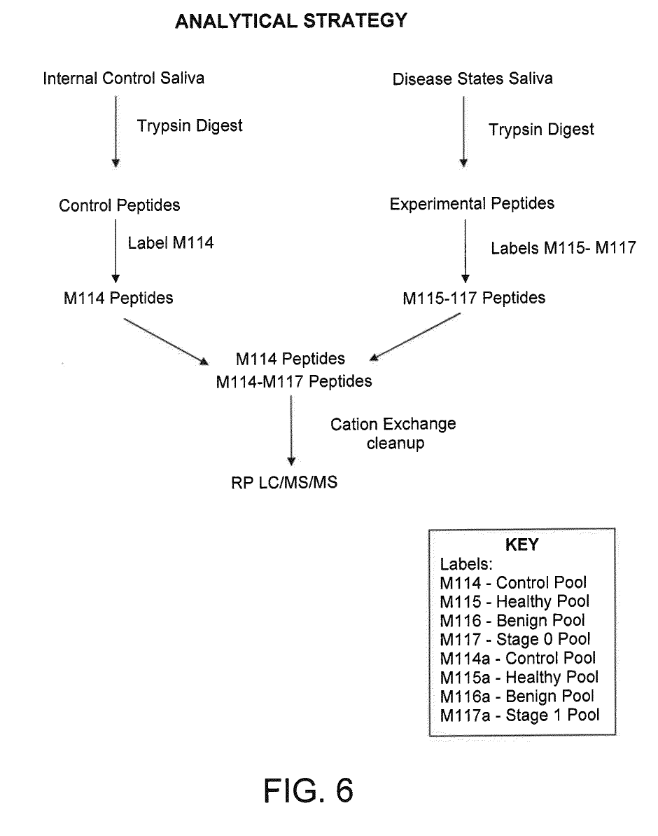

The precipitate was resuspended and treated according to the manufacturer's instructions. The treatment included blocking cysteine residues with methylmethane thiosulphate (MMTS) and trypsin digestion (FIG. 6). The peptides generated during the digestion were labeled with specifically coded iTRAQ™ reagents. The labeled peptides from each of the saliva samples were then combined and partially purified by a combination of cation exchange chromatography and desalting on a reverse phase column. The desalted and concentrated peptide mixtures were analyzed by MS. Before MS, an aliquot of the peptide mixture was separated by HPLC on a C18 75-μ×10-cm reverse phase capillary column (Vydac 218MS3.07510). A linear gradient of 2% to 50% acetonitrile in 0.1% formic acid, over 180 minutes followed by 40 minutes wash with 2% acetonitrile was used to elute the peptides directly into the mass spectrometer for tandem MS analysis. The QSTAR™ operates in an information-dependent acquisition mode, which detects peptides by TOF-MS and then fragments them by collision-induced dissociation in the tandem MS mode. The accumulated tandem MS spectra were analyzed by ProQuant™ (Applied Biosystems).

Table 9 shows the results of the experiment using pooled saliva samples. Overall 70 proteins were identified at greater than 99% confidence level (two or more peptides sequenced at >99% confidence interval) and 209 proteins at greater than 95% confidence level (at least one peptide sequenced at >99% confidence interval). The internal standards and their resultant protein profiles were compared and produced similar results. Likewise, the healthy and benign subject spectra between the two runs were also comparable demonstrating reliable and reproducible data for additional spectral comparisons across the two individual runs. As illustrated in Table 10, the healthy subjects were labeled with a 115 marker, the benign subjects with a 116 marker, and the cancer groups with the 117 marker. Comparisons are listed in Table 10. A list of candidate up- and down-regulated proteins is listed in Tables 10 and 12. It is believed that the protein markers ubiquitin (Acc. No. P68197) and cytochrome p450 (Acc. No. Q9GQM9), listed in Tables 11 and 12, respectively, have not been previously associated with breast tumors.

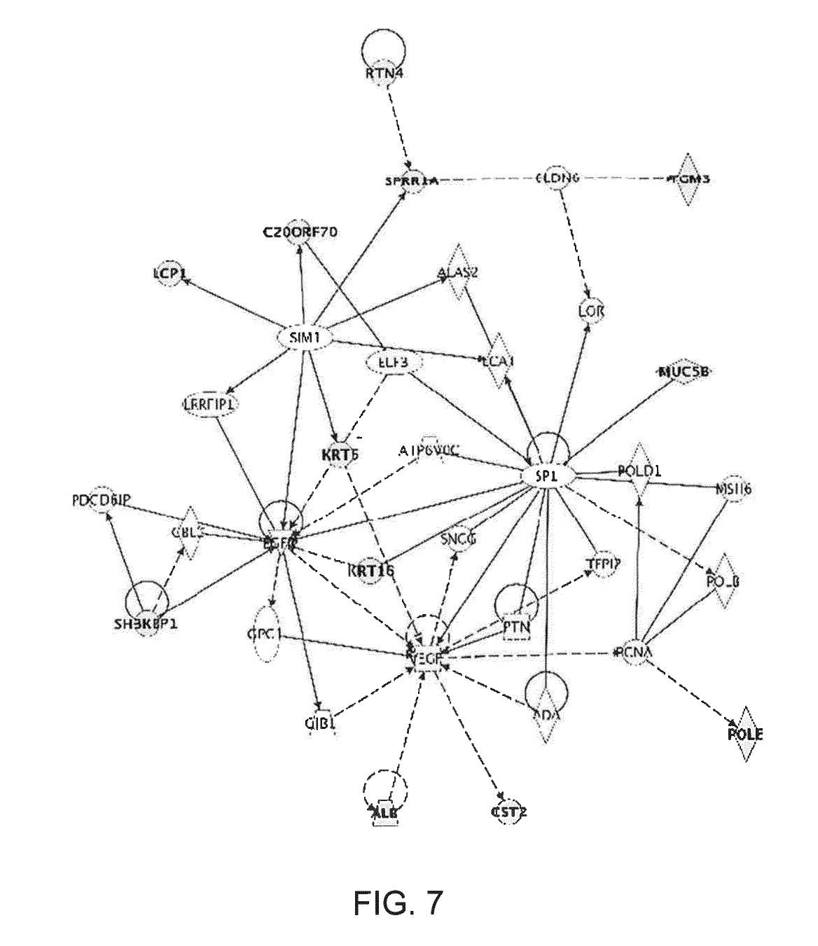

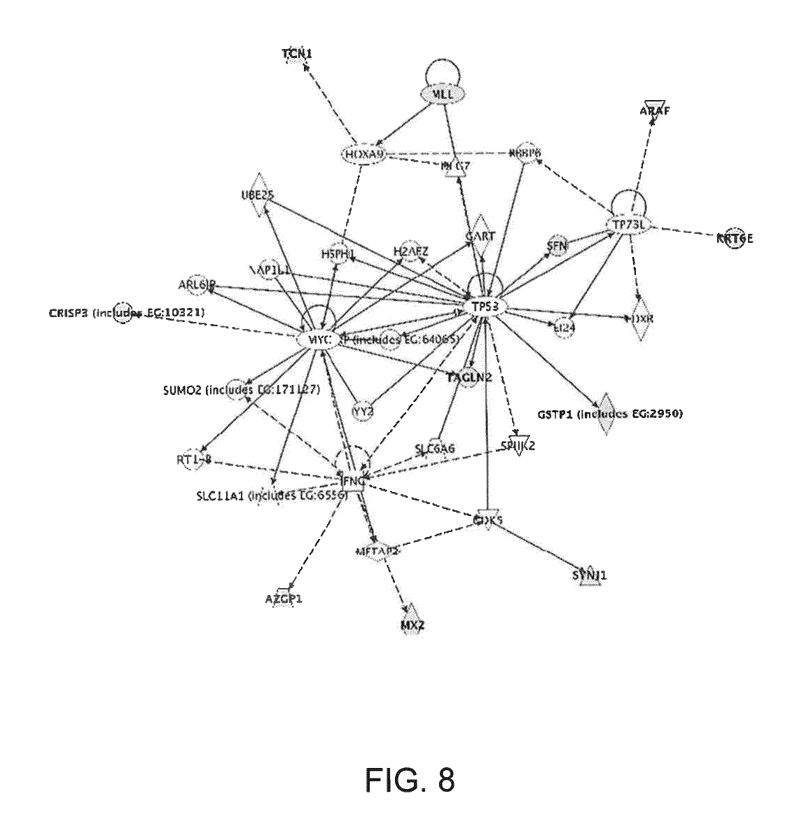

The proteins were entered into Ingenuity™ software, pathway analysis software application that enables researchers to model, analyze, and understand the complex biologic and chemical systems at the core of cancer research. The results of the analyses are in FIGS. 7 and 8. FIG. 7 shows an association with the epidermal growth factor (EGF) pathway, which is at the heart of carcinogenesis, whereas FIG. 4 suggests associations with the ubiquitin pathway. As illustrated, the proteins are associated with varying pathways that are common to both the ductal epithelia of the breast and the ductal epithelia of the salivary glands. Additionally, these same proteins have been found to be either up- or down-regulated in MCF-7 and SKBR-3 cancer cell lines. Interestingly, the results identified proteins that are both up- or down-regulated, have varying cellular functions, and have been validated in cell studies to be altered in the presence of carcinoma of the breast.

### Example 11

### Cytochrome P450 as a Marker for Breast Cancer

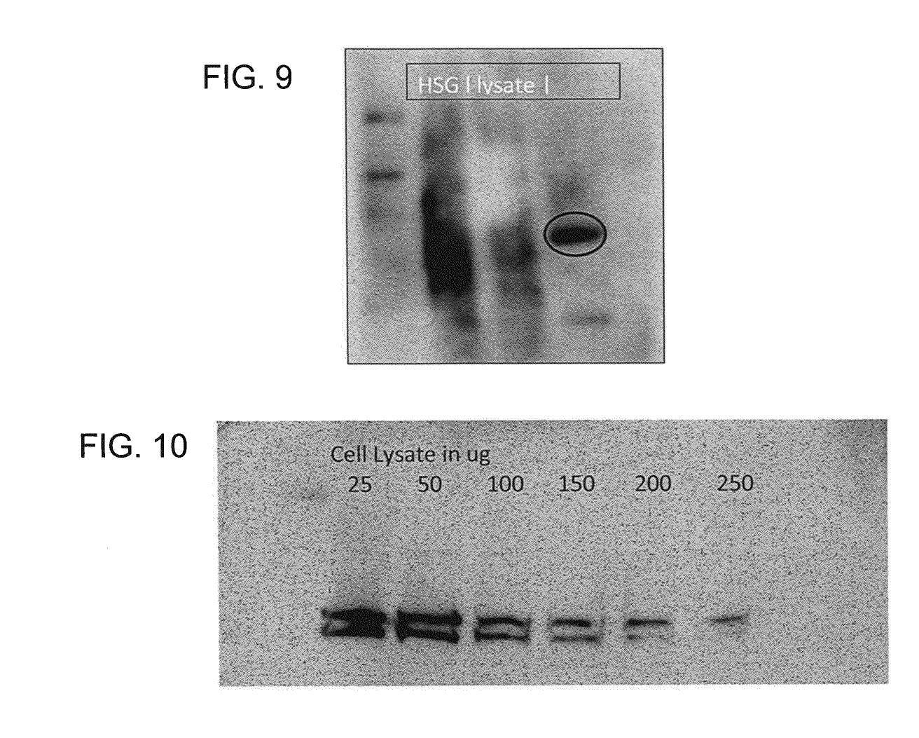

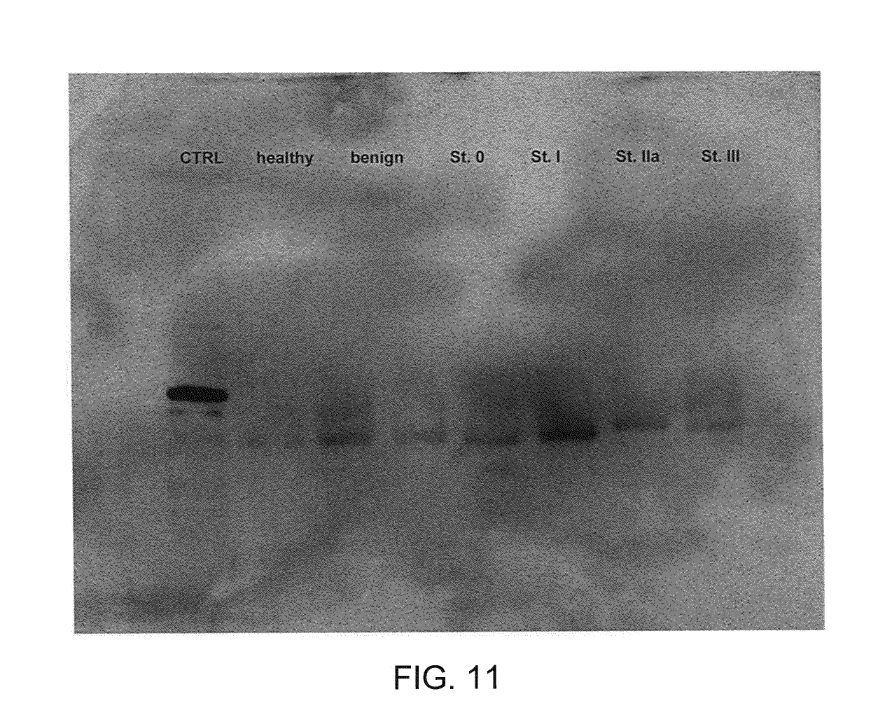

Referring now to FIG. 9, a western blot was prepared demonstrating the presence of cytochrome p450 in human salivary gland cell lysate, SKBR3 cell lysate and in saliva. The cytochrome p450 band in the saliva sample is circled in FIG. 9. A western blot of a SKBR3 cell lysate control dilution series with cytochrome p450 antibody is shown in FIG. 10. Cell lysate dilutions range from 25 to 250 μg. FIG. 11 is a western blot obtained with control (stage IV breast cancer), healthy and benign salivas, and with salivas from stages 0, I, IIa and III breast cancer patients using anti-cytochrome p450 antibody.

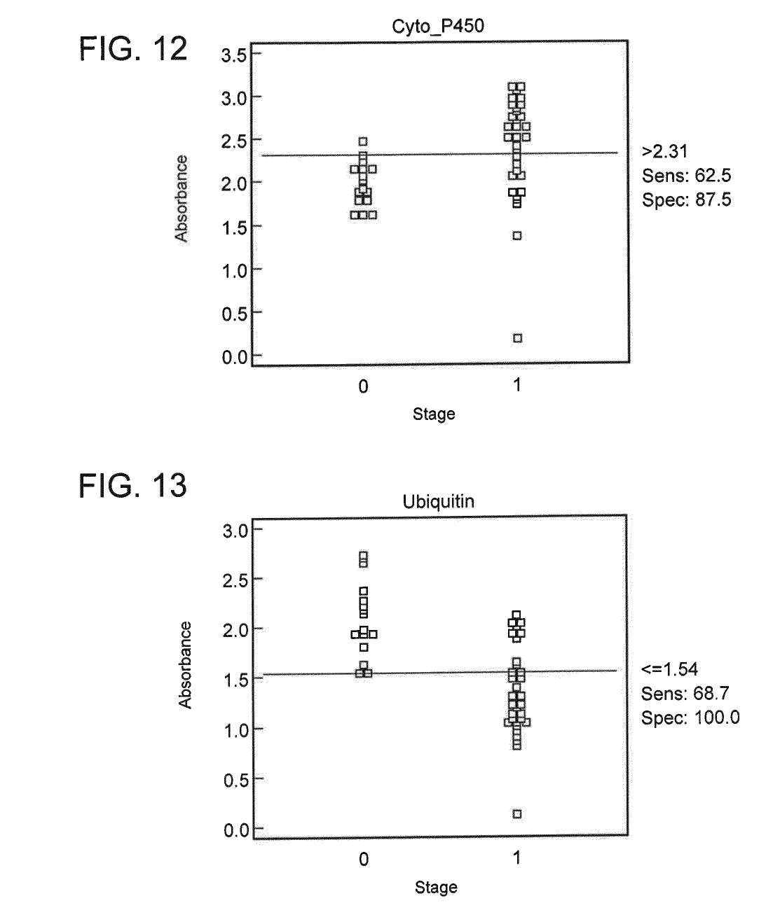

FIGS. 12-17 are box plots showing that the saliva samples' 2-step indirect ELISA data correlates to the health status of the patient; strength of the test ability is denoted as sensitivity and specificity. Patient saliva samples were centrifuged at 14,000×g for 10 minutes at 4° C. and 25 microliters of cleared saliva was mixed with 175 microliters of PBS (pH 8). 200 microliters of each diluted sample was added in duplicate to amine-binding 96-well assay plates (Pierce-Amine-binding Maleic Anhydride 96-well plates; no. 15110) and the plates were incubated at 37° C. for 1.5 hours. The saliva samples were decanted from the plates and 300 microliters of 5× Blocker BSA was added to each well and the plate was incubated at 37° C. for 1 hour. The wells were then washed 3× with 300 microliters Tris-Cl buffer (pH 7.4) and 200 microliters of the primary antibody (diluted 1:2000 in Tris-Cl, 0.05% BSA) was added. The plates were incubated at 37° C. for 1 hour to allow antibody binding and then the wells were washed 3× with 300 microliters Tris-Cl buffer (pH 7.4). 200 microliters of the secondary antibody-alkaline peroxidase (AP) conjugate (diluted 1:20000 in Tris-Cl, 0.05% BSA) was added to each well and the plates were incubated at 37° C. for 1 hour. The wells were washed 3× with 300 microliters Tris-Cl buffer (pH 7.4) and 100 microliters of detection reagent, 1-step p-Nitrophenyl Phosphate (PNPP: Pierce) substrate solution for AP was added to each well and the plates were incubated at room temperature, on a shaker for 15 minutes. The color producing reaction was stopped by the addition of 2N NaOH and the absorbance at 405 nm was read using a spectrophotometer.

Referring now to FIG. 12, ROC curve analysis was performed with the candidate biomarker cytochrome p450 using 2-step indirect-ELISA assays. In brief, samples were done in duplicate and the absorbance values were averaged for each ELISA. The test-values (absorbance) were listed alongside of Disease Status for each sample. ROC curve analysis was performed on MedCalc™ software following normal procedure (noted in Help Menu of MedCalc™). The software automatically picked the cut-off point with the best sensitivity and specificity. The results are summarized in Tables 8A-8D for healthy vs. stage 0-IV cancer (Table 8A), healthy vs. diseased (benign to stage IV) (Table 8B), benign vs. stage 0-TV cancer (Table 8C), and healthy vs. benign (Table 8D). These data demonstrate how staging of breast cancer may be conducted using cytochrome p450 and other protein markers identified herein. Data from benign vs. cancer (stages 0 to IV) obtained with cytochrome p450 are shown in FIG. 12, and demonstrates up-regulation of cytochrome p450 between benign and cancer samples. A box plot showing the relationship between healthy and benign samples (summarized in Table 8D) is shown in FIG. 15.

### Example 12

### Ubiquitin as a Marker for Breast Cancer

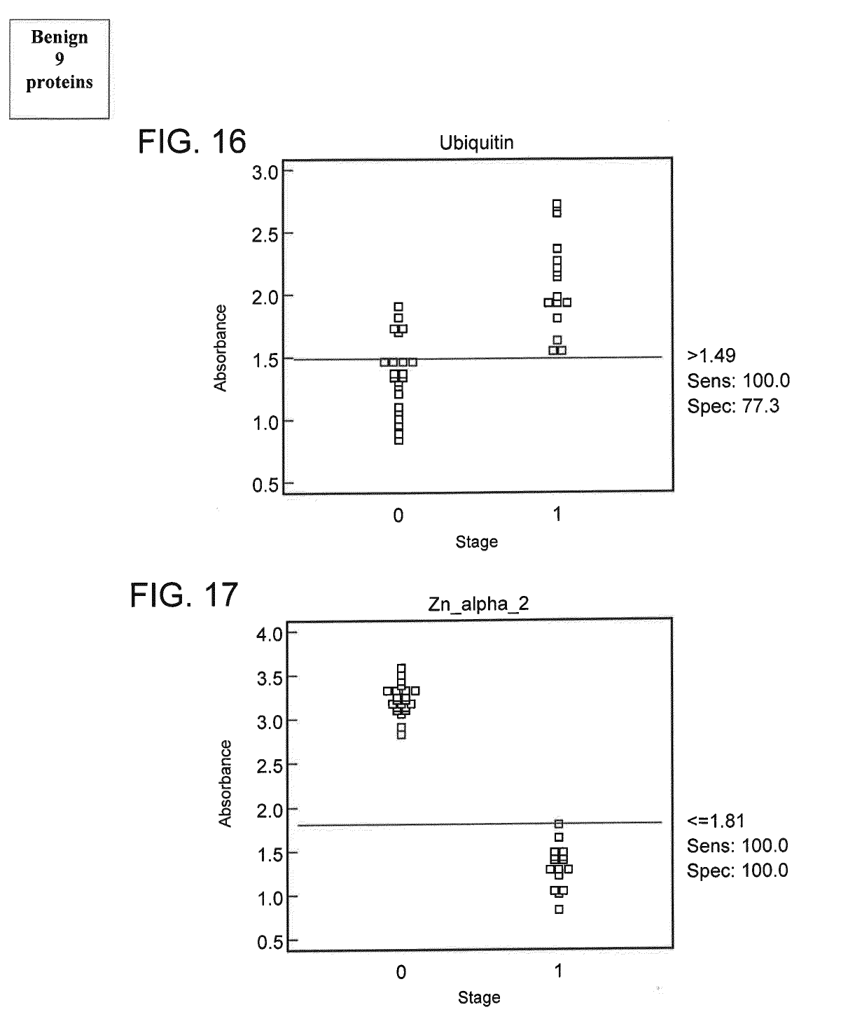

ROC curve analysis was performed with the candidate biomarker ubiquitin using 2-step indirect ELISA assays, as described above with respect to cytochrome p450. The results are summarized in Tables 8A-8D for healthy vs. stage 0-IV cancer (Table 8A), healthy vs. diseased (benign to stage IV) (Table 8B), benign vs. stage 0-TV cancer (Table 8C), and healthy vs. benign (Table 8D). Data from benign vs. cancer (stages 0 to IV) obtained with ubiquitin are presented as a box plot in FIG. 13 which demonstrates that ubiquitin is downregulated between benign and cancer samples. A box plot showing the relationship between healthy and benign samples (summarized in Table 8D) for ubiquitin is shown in FIG. 16.

### Example 13

### Zinc Alpha 2 as a Marker for Breast Cancer

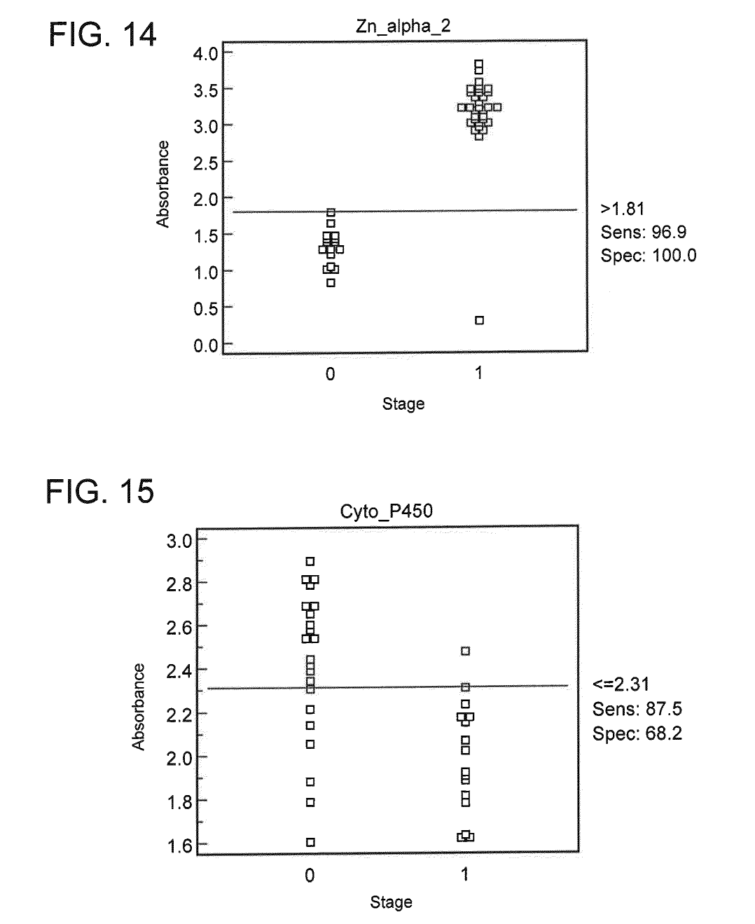

ROC curve analysis was performed with the candidate biomarker zinc alpha 2 using 2-step indirect ELISA assays as described above with respect to cytochrome p450. The results are summarized in Tables 8A-8D for healthy vs. stage 0-IV cancer (Table 8A), healthy vs. diseased (benign to stage IV) (Table 8B), benign vs. stage 0-IV cancer (Table 8C), and healthy vs. benign (Table 8D). Data from healthy vs. cancer (stages 0 to TV) obtained with zinc alpha 2 are shown in FIG. 14, and demonstrates up-regulation of zinc alpha 2 between benign and cancer samples. A box plot showing the relationship between healthy and benign samples (summarized in Table 8D) is shown in FIG. 17.

### Example 14

### Detection of Increased Risk of Breast Cancer

In some embodiments, a testing method is employed for diagnostic and/or prognostic evaluation of patients who might be at increased risk of having or developing breast cancer. Cytochrome p450 or ubiquitin levels may be measured by assaying its enzymatic activity. For example, ubiquitination is an enzymatic, protein post-translational modification (PTM). The process of marking a protein with ubiquitin (ubiquitylation or ubiquitination) consists of a series of steps including activation of ubiquitin. Ubiquitin is activated in a two-step reaction by an E1 ubiquitin-activating enzyme in a process requiring ATP as an energy source. The initial step involves production of a ubiquitin-adenylate intermediate. The second step transfers ubiquitin to the E1 active site cysteine residue, with release of AMP. This step results in a thioester linkage between the C-terminal carboxyl group of ubiquitin and the E1 cysteine sulfhydryl group.

The cytochrome P450 family (officially abbreviated as CYP) is a large and diverse group of enzymes. The function of most CYP enzymes is to catalyze the oxidation of organic substances. The substrates of CYP enzymes include metabolic intermediates such as lipids, steroidal hormones as well as xenobiotic substances such as drugs.

In some embodiments, cytochrome p450 and ubiquitin levels are measured through the use of immunoassays, in any of a variety of different formats including, but not limited to, radioimmunoassay (RIA), electroimmunoassay, enzyme-linked immunoassay systems (ELISA), immunonephelometry or immunoturbidimetry, immunochromatography, immunoprecipitation, immunofluormetry, as well as those immunoassays that are less often used for high throughput, such as, but not limited to Western blots, immunodiffusion, immunoelectrophoresis, immunohistochemistry. Thus, it can be appreciated that a wide variety of cytochrome p450 or ubiquitin measurement technologies are currently available to implement various embodiments of the disclosed methods to identify patients at increased risk of breast cancer by determining the relative levels of cytochrome p450 or ubiquitin, alone or in combination with each other and/or additional breast cancer biomarkers, in the patient saliva as an indication of increased risk of existing or developing breast cancer in the patient. Alternatively, any other suitable technology for measuring cytochrome p450 or ubiquitin levels may be used in these methods. Examples of other breast cancer biomarkers that may be assayed in combination with cytochrome p450 and/or ubiquitin are described herein, and in co-pending U.S. patent application Ser. No. 12/678,686 or in U.S. Pat. Nos. 6,294,349, 6,670,141, 6,972,180; published U.S. Patent Application Nos. 20020015964 and 20040033613; and PCT Patent Application Publication Nos. WO 2009/039023 and WO 2000/052463. Although the present discussion of antibody production and use focuses primarily on immunoassays for cytochrome p450 and ubiquitin, it should be understood that antibodies for other protein markers may be obtained similarly and assayed using similar techniques.

### Example 15

### Detection of Cytochrome p450 or Ubiquitin

In some embodiments, a testing method is performed by utilizing pre-packaged diagnostic kits comprising a cytochrome p450 or ubiquitin nucleotide sequence, a cytochrome p450 or ubiquitin protein or peptide, and/or a cytochrome p450 or ubiquitin antibody reagent described herein, which may be conveniently used, e.g., in clinical settings, to identify patients at risk for breast cancer. Any suitable clinical assay currently used or approved for use in determining human or animal cytochrome p450 or ubiquitin levels in bodily fluids, preferably saliva, may be used in some embodiments of the disclosed methods. Such assays may also be used to diagnose, stage and follow breast cancer patients to identify individuals at risk for breast cancer or progression.

### Example 16

### Combination of Cytochrome p450 and Ubiquitin Assays

In some cases, a patient suspected of being at some risk of having or developing breast cancer has a saliva sample, or aliquots thereof, collected which is subjected to both a cytochrome p450 and a ubiquitin assay. Should one or both of these assays reveal that the patient's saliva cytochrome p450 level is decreased or that the patient's ubiquitin saliva levels are increased above the normal range, then it can be assumed that the patient is at heightened risk of having or developing breast cancer. For some applications assays for additional protein markers either described herein or known to be breast cancer biomarkers, are performed in combination with cytochrome p450 and/or ubiquitin assays to provide a more robust diagnosis or prognosis. For example, in some applications assays of cytochrome p450, ubiquitin and one or more of S100, profilin-1, Za2G and adiponectin are performed on a patient's saliva, for making a diagnosis. In some combinations, an assay for HER2/neu is also performed, using any suitable assay method.

In certain embodiments, a method for detecting cytochrome p450 or ubiquitin in a sample is provided which comprises contacting sample proteins or peptides from a sample suspected of containing cytochrome p450 or ubiquitin with at least a first antibody that binds to a cytochrome p450 or ubiquitin protein or peptide, under conditions effective to allow the formation of immune complexes, and detecting the immune complexes thus formed. In some cases, the sample proteins contacted are located within a cell, while in others, the sample proteins are separated from a cell prior to contact. Thus, in some embodiments an immunoassay detection kit is provided that comprises at least a first antibody (e.g., a monoclonal antibody) capable of binding to a cytochrome p450 or ubiquitin protein or peptide and a detection reagent, along with testing instructions. In some instances, the immunoassay detection kit also includes an unrelated protein or peptide for use as a control, and/or a second antibody that binds to the first antibody. In some embodiments, the kit contains the necessary components for testing saliva for cytochrome p450 or ubiquitin levels, to determine whether the individual is at increased risk breast cancer. Test kits are further described below.

### Example 17

### Test Kits

Some embodiments of the diagnostic methods for identifying a patient at risk for developing breast cancer include determining the relative amount of cytochrome p450 and/or ubiquitin present within the saliva of the patient, wherein a decrease in cytochrome p450 or an increase in ubiquitin in comparison to a sample from a normal subject is indicative of the patient having, or being at increased risk of developing, breast cancer. All the essential materials and reagents required for conducting such methods may be assembled together in a kit to facilitate the rapid and easy identification of patient as being at risk for breast cancer. With such a kit, the user determines whether a particular sample of a patient's saliva contains a level of cytochrome p450 that is below the normal range or, and may also determine whether a particular sample of a patients saliva contains a level of ubiquitin that is above the normal range. These assays may be done as two distinct assays, using, for example, two test strips. One test strip would contain an immunoassay technology for assaying cytochrome p450 and the other test strip ubiquitin. Alternatively, in some embodiments both tests are performed using a single test strip that contains both a detection system for cytochrome p450 and ubiquitin.

For example, in some embodiments an immunoassay detection kit is provided that comprises at least a first antibody (e.g., a monoclonal antibody) capable of binding to a cytochrome p450 or ubiquitin protein or peptide and a detection reagent, along with testing instructions. In some instances, the immunoassay detection kit also includes an unrelated protein or peptide for use as a control, and/or a second antibody that binds to the first antibody. In some embodiments, the kit contains the necessary components for testing a bodily fluid for cytochrome p450 or ubiquitin, to determine whether the individual is at increased risk of breast cancer.

A test kit may have a single container means, or it may have individual containers for each reagent. When the components of the kit are provided in one or more liquid solutions, the liquid solution is an aqueous solution, with a sterile aqueous solution being particularly preferred. The components of the kit may also be provided in dried or lyophilized forms. When reagents or components are provided as a dried form, reconstitution generally is by the addition of a suitable solvent. It is envisioned that the solvent also may be provided in another container means. It is envisioned that the solvent may also be provided in another container means. In some cases, the container means also includes at least one vial, test tube, flask, bottle, syringe or other container means, into which the test reagent formulation are placed, preferably, suitably allocated. In some instances, a kit also comprises a second container means for containing a sterile, pharmaceutically acceptable buffer or other diluent. In which case, the container means may itself be a syringe, pipette, or other such like apparatus even applied to and mixed with the other components of the kit. Irrespective of the number or type of containers employed in a kit, in some embodiments the kit also includes, or is packaged with, an instrument for signal detection and analysis. In some embodiments, a kit includes a means for containing the vials in close confinement for commercial sale such as, e.g., an injection or blow-molded plastic container in which the desired vials are retained. In most embodiments, instructions for use of the kit components are included. For some applications, an above-described kit may also include test materials for detecting and quantitating one or more additional breast cancer biomarkers identified herein or known to be a marker for breast cancer.

TABLE 1

| Comparison | Up Regulated | Down Regulated | Total Markers |
|---|---|---|---|
| Benign vs. Healthy | 14 | 9 | 23 |
| Cancer vs. Healthy | 20 | 12 | 32 |
| Cancer vs. Benign | 17 | 11 | 28 |
| Totals | 51 | 32 | 83 |

TABLE 2
Benign vs. Healthy

| | | | | |
|---|---|---|---|---|
| | | | p | |
| Accession | Protein Name | Ratio | Value | Gene ID. |

Up - Regulated Proteins in Benign

| | | | | |
|---|---|---|---|---|
| P06733 | Alpha enolase | 1.4204 | 0.0006 | ENOA |
| P04083 | Annexin Al | 1.6282 | 0.0047 | ANXA1 |
| P05109 | Calgranulin A | 1.9393 | 0.0001 | S10A8 |
| P06702 | Calgranulin B | 1.6297 | 0.0002 | S10A9 |
| Q9UBC9 | Cornifin beta | 2.1353 | 0.0000 | SPRR3 |
| P01036 | Cystatin S precursor | 1.2584 | 0.0027 | CYTS |
| P01877 | Ig alpha-2 chain C region | 1.2781 | 0.0213 | 1GHA2 |
| P01871 | Ig mu chain C region | 1.256 | 0.0196 | MUC |
| P13646 | Keratin, type I cytoskeletal 13 | 1.3184 | 0.0180 | K1CM |
| Q9QWL7 | Keratin, type I cytoskeletal 17 | 2.6008 | 0.0018 | K1CQ |
| P04264 | Keratin, type II cytoskeletal 1 | 1.4504 | 0.0002 | K2C1 |
| P48666 | Keratin, type II cytoskeletal 6C | 2.0979 | 0.0003 | K2C6C |
| Q9HC84 | Mucin 5B precursor | 1.4306 | 0.0001 | MUC5B |
| P05164 | Myeloperoxidase precursor | 1.8949 | 0.0015 | PERM |

Down-Regulated Proteins in Benign

| | | | | |
|---|---|---|---|---|
| P28325 | Cystatin D precursor | 0.817 | 0.0455 | CYTD |
| P18510 | Interleukin-1 receptor antagonist protein precursor | 0.7484 | 0.0312 | IL1RA |
| P22079 | Lactoperoxidase precursor | 0.7408 | 0.0137 | PERL |
| P80188 | Neutrophil gelatinase-associated lipocalin precursor | 0.7971 | 0.0289 | NGAL |
| P31151 | 5100 calcium-binding protein A7 | 0.4737 | 0.0054 | S10A7 |
| P04745 | Salivary alpha-amylase precursor | 0.8245 | 0.0023 | AMYS |
| P02787 | Serotransferrin precursor | 0.6968 | 0.0000 | TRFE |
| P02768 | Serum albumin precursor | 0.6922 | 0.0000 | ALBU |
| Q96DR5 | Short palate, lung and nasal epith. Ca assoc.protein 2 | 0.7798 | 0.0170 | SPLC2 |

TABLE 3
Cancer vs. Healthy

| | | | | | | | |
|---|---|---|---|---|---|---|---|
| Accession | | | | | Reported | Blood | Tissue |
| Number | Protein Name | Ratio | P Value | Gene ID | Function | (Ref.) | (Ref.) |

Up-Regulated Proteins in Cancer Saliva

| | | | | | | | |
|---|---|---|---|---|---|---|---|
| Q9DCT1 | Aldo-keto reductase | 1.44 | 0.0264 | AK1E1 | Detox & reduction | — | 25 |
| P04083 | Annexin A1 | 3.06 | 0.0001 | ANXA1 | Membrane associated protein | 30 | 25 |
| P05109 | Calgranulin A | 2.18 | 0.0001 | S10A8 | Cell adhesion & communication | 30 | — |
| P06702 | Calgranulin B | 1.87 | 0.0001 | S10A9 | Cell adhesion & communication | 30 | — |
| P23280 | Carbonic anhydrase VI | 1.52 | 0.0003 | CAH6 | Energy/metabolism | 30 | 27 |
| Q9UBC9 | Cornifin beta | 1.82 | 0.0001 | SPRR3 | Indicator of tissue damage | — | — |
| P13646 | Cytokeratin 13 | 6.56 | 0.0001 | K1CM | Intracytoplasmatic cytoskeleton protein | 30 | — |
| P19013 | Cytokeratin 4 | 6.50 | 0.0019 | K2C4 | Intracytoplasmatic cytoskeleton protein | 30 | 25 |
| P48666 | Cytokeratin 6C | 4.41 | 0.0001 | K2C6C | Intracytoplasmatic cytoskeleton protein | — | — |
| P01040 | Cystatin A | 2.00 | 0.0014 | CYTA | Protein degradation & inhibitor | 30 | 25 |
| P01036 | Cystatin SA-III | 1.20 | 0.0115 | CYTS | Protein degradation & inhibitor | — | — |
| Q01469 | Epid. Fatty acid-binding prot. | 2.1 | 0.0362 | FABP4 | Protein with binding functions | 30 | — |
| P01857 | Ig gamma-1 chain C region | 1.44 | 0.0034 | IGHG1 | Immunoresponse | — | — |
| P01871 | Ig mu chain C region | 1.51 | 0.0011 | MUC | Immunoresponse | — | — |
| P06870 | Kallikrein 1 precursor | 1.23 | 0.0425 | KLK1 | Serine protease | | |
| P02788 | Lactoferrin | 1.58 | 0.0001 | TRFL | Inhibits G1 CDK's, mod. NK activity | 30 | 26 |
| Q9HC84 | Mucin 5B | 1.68 | 0.0001 | MUC5B | Cell adhesion & communication | — | 36 |
| P05164 | Myeloperoxidase precursor | 2.72 | 0.0005 | PERM | Defense Immunoresponse | 30 | — |
| P31151 | S100 calcium-binding protein | 2.05 | 0.0001 | S100P | Calcium binding protein | 30 | 25 |
| P31025 | Von Ebner's gland protein (lipocalin) | 1.26 | 0.0043 | VEGP | Inflammation | — | 25 |

Down-Regulated Proteins in Cancer Saliva

| | | | | | | | |
|---|---|---|---|---|---|---|---|
| Q8N4F0 | Bact. Perm.-increasing prot.-1 | 0.80 | 0.0004 | BPIL1 | Transport | 30 | — |
| P04264 | Cytokeratin 1 | 0.61 | 0.0001 | K2C1 | Intracytoplasmatic cytoskeleton protein | — | 25 |
| P01034 | Cystatin C | 0.72 | 0.0187 | CYTC | Inhibitor of cysteine proteases | — | 31 |
| P28325 | Cystatin D precursor | 0.68 | 0.001 | CYTD | Protein degradation & inhibitor | — | — |
| P00738 | Haptoglobin | 0.83 | 0.0023 | HPT | Indicator of tissue damage and necrosis | 30, 34 | — |
| P22079 | Lactoperoxidase | 0.82 | 0.0388 | PERL | Transport | — | 33 |
| P01833 | Poly-IG receptor protein | 0.86 | 0.0234 | PIGR | Immunoresponse | | |
| P07737 | Profilin-1 | 0.68 | 0.0135 | PROF1 | Cytoskeleton associated | — | 25 |
| P02768 | Serum albumin precursor | 0.73 | 0.0001 | ALBU | Transport | 30 | 27 |
| Q96DR5 | Short palate, lung and nasal | 0.61 | 0.0001 | SPLC2 | Immune response & detox. | — | 32 |
| | epith. carc. assoc. protein 2 | | | | | | |
| P02787 | Transferrin | 0.72 | 0.0001 | TRFE | Surface antigen assoc. with growth | 34 | — |
| P25311 | Zinc-alpha-2-glycoprotein | 0.84 | 0.0009 | ZA2G | Signaling | — | 29 |

TABLE 4
Cancer vs. Benign

| | | | | |
|---|---|---|---|---|
| Accession | Protein Name | Ratio | p Value | Gene ID. |

Up-Regulated Proteins in Cancer

| | | | | |
|---|---|---|---|---|
| Q9HC84 | Mucin 5B precursor | 1.16 | 0.0046 | MUC5B |
| P80188 | lipocalin precursor | 1.18 | 0.0443 | NGAL |
| P01871 | Ig mu chain C region | 1.19 | 0.0231 | MUC |
| P04745 | Salivarv alpha-amylasc precursor | 1.19 | 0.001 | AMYS |
| P23280 | Carbonic anhydrase VI precursor | 1.29 | 0.0293 | CAH6 |
| P02788 | Lactotransferrin precursor | 1.30 | 0.0113 | TRFL |
| P05164 | Myeloperoxidase precursor | 1.41 | 0.0487 | PERM |
| P01857 | Ig gamma-1 chain C region | 1.43 | 0.0004 | IGHG1 |
| Q9DCT1 | Aldo-keto reductase | 1.47 | 0.018 | AK1E1 |
| P31025 | Von Ebner's gland protein | 1.55 | 0.0005 | VEGP |
| P04083 | Annexin Al | 1.86 | 0 | ANXA1 |
| P10599 | Thioredoxin | 1.93 | 0.03 | THIO |
| P01040 | Cystatin A | 1.95 | 0.0056 | CYTA |
| P48666 | Keratin, type II cytoskeletal 6C | 2.07 | 0 | K2C6C |
| P31151 | S100 calcium-binding protein A7 | 4.08 | 0.0005 | S10A7 |
| P13646 | Keratin, type I cytoskeletal 13 | 4.76 | 0 | K1CM |
| P19013 | Keratin, type II cytoskeletal 4 | 5.59 | 0.0013 | K2C4 |

Down-Regulated Proteins in Cancer

| | | | | |
|---|---|---|---|---|
| P04264 | Keratin, type II cytoskeletal 1 | 0.42 | 0 | K2CI |
| Q9QWL7 | Keratin, type I cytoskeletal 17 | 0.58 | 0.001 | K1CQ |
| P01034 | Cystatin C precursor | 0.67 | 0.0246 | CYTC |
| P13796 | L-plastin | 0.69 | 0.0145 | PLSL |
| P06733 | Alpha enolase | 0.73 | 0.0106 | ENOA |
| P01833 | Polymeric-Ig receptor | 0.76 | 0.0002 | PIGR |
| P12273 | Prolactin-inducible protein | 0.77 | 0.0012 | PIP |
| | precursor | | | |
| Q95DR5 | Short palate, lung and nasal | 0.78 | 0.0219 | SPLC2 |
| | epithelium carcinoma associated | | | |
| | protein 2 precursor | | | |
| P07477 | Trypsin I precursor | 0.82 | 0.0196 | TRY1 |
| P28325 | Cystatin D precursor | 0.83 | 0.0155 | CYTD |
| Q9UBC9 | Small proline-rich protein 3 | 0.84 | 0.0228 | SPRR3 |

TABLE 5
Low risk Subjects vs. Stage 0 Cancer Subjects
Level 3 - Changes greater than 2-fold from good quality signal that pass a visual inspection

| | | | | | | Swiss | | Predicted | | | | Fold- |
|---|---|---|---|---|---|---|---|---|---|---|---|---|
| SSP | Acc. No. | Type | Lane | Protein Name | Level | Prot ID | Locus | MW | MW | Signal | Change | Change |
| 5602 | A13920 | CONTROL | 25 | Annexin I | 3 | P46193 | 301 | 38 | 40 | 0 | − | 26.65 |
| 2501 | A14020 | CONTROL | 5 | Annexin II | 3 | P07355 | 302 | 36 | 39 | 1 | − | div/0 |
| 6903 | A92820 | CONTROL | 27 | Apaf-1 | 3 | O14727 | 317 | 130 | 137 | 1 | − | div/0 |
| 5702 | R10820 | CONTROL | 22 | BAG-1-58KD | 3 | Q99933 | 573 | 50/46/33 | 58 | 1 | − | 12.90 |
| 6601 | C41720 | CONTROL | 27 | Calreticulin-51KD | 3 | P14211 | 12317 | 60 | 51 | 1 | − | div/0 |
| 6702 | C41720 | CONTROL | 27 | Calreticulin-54KD | 3 | P14211 | 12317 | 60 | 54 | 1 | − | div/0 |
| 9703 | C20820 | CANCER | 30 | E-Cadherin | 3 | P12830 | 999 | 120 | 120 | 1 | − | div/0 |
| 5901 | N38620 | CONTROL | 23 | eNOS/NOS Type III | 3 | P29474 | 4846 | 140 | 140 | 1 | − | div/0 |
| 3901 | C26220 | CONTROL | 9 | g-Catenin | 3 | Q15151 | 3728 | 82 | 82 | 1 | − | div/0 |
| 6401 | G16720 | CONTROL | 28 | GRB2 | 3 | P29354 | 81504 | 24 | 24 | 0 | + | 3.09 |
| 8401 | G16720 | CONTROL | 33 | GRB2-24KD | 3 | P29354 | 81504 | 24 | 24 | 1 | + | 3.86 |
| 8501 | G16720 | CONTROL | 33 | GRB2-28KD | 3 | P29354 | 81504 | 24 | 28 | 0 | + | 10.15 |
| 1601 | M93620 | CANCER | 5 | Mint3/X11g-61KD | 3 | O88888 | 9546 | 61 | 61 | 0 | + | 4.65 |
| 4904 | M94120 | CONTROL | 14 | MSH3-138KD | 3 | P20585 | 4437 | 127 | 138 | 1 | − | div/0 |
| 3607 | P35220 | CONTROL | 7 | PP1 | 3 | P08129 | 5499 | 36 | 41 | 1 | − | 2.18 |
| 202 | R23520 | CANCER | 2 | Ral A | 3 | P11233 | 5898 | 24 | 26 | 1 | − | 21.01 |
| 2101 | S10520 | CANCER | 8 | Spot 14 | 3 | Q92746 | 7069 | 17 | 17 | 0 | + | 2.00 |
| 2601 | T57120 | CONTROL | 2 | Tau-53KD | 3 | P10636 | 4137 | 50-68 | 53 | 1 | + | 3.02 |

TABLE 6
Low risk Subjects vs. Stage 0 Cancer Subjects
Level 2 - Changes greater than 2-fold from good quality signals that do not pass a visual inspection

| | | | | | | Swiss | | Predicted | | | | Fold- |
|---|---|---|---|---|---|---|---|---|---|---|---|---|
| SSP | Acc. No. | Type | Lane | Protein Name | Level | Prot ID | Locus | MW | MW | Signal | Change | Change |
| 7503 | | CANCER | 20 | alpha-Tubulin | 2 | 0 | 0 | 55 | 58 | 1 | − | 1.92 |
| 5504 | F14220 | CONTROL | 23 | basic FGF-25KD | 2 | P09038 | 2246 | 18-24 | 25 | 1 | + | 3.30 |
| 6602 | C80420 | CONTROL | 29 | CD38 | 2 | P28907 | 952 | 46 | 41 | 1 | − | div/0 |
| 4601 | C45820 | CONTROL | 17 | CDC25B-53KD | 2 | O43550 | 994 | 63 | 53 | 0 | + | 2.20 |
| 7702 | F19720 | CONTROL | 30 | fyn | 2 | P06241 | 2534 | 59 | 55 | 0 | + | 3.51 |
| 4501 | G59720 | CONTROL | 14 | GST-p | 2 | P09211 | 2950 | 23 | 25 | 1 | + | 3.89 |
| 7404 | H10520 | CANCER | 20 | KNP-1/HES1 | 2 | 0 | 0 | 28 | 28 | 1 | − | 4.38 |
| 7601 | L15620 | CONTROL | 31 | lck-53KD | 2 | P06239 | 3932 | 56 | 53 | 1 | + | 3.67 |
| 2301 | N13320 | CANCER | 8 | NES1 | 2 | O43240 | 5655 | 30 | 28 | 0 | − | 2.00 |
| 4201 | N42420 | CONTROL | 20 | NTF2 | 2 | 0 | 0 | 15 | 15 | 0 | + | 1.89 |
| 3402 | P11920 | CONTROL | 9 | p24 | 2 | P97799 | 22360 | 24 | 24 | 1 | + | 4.48 |
| 5608 | A36520 | CONTROL | 26 | Phospho-Akt (S473) | 2 | P31749 | 207 | 59 | 54 | 0 | + | 3.89 |
| 3610 | P67920 | CONTROL | 7 | PKBa/Akt-53KD | 2 | P31749 | 207 | 59 | 53 | 1 | + | 2.56 |
| 7608 | P97220 | CANCER | 21 | PKR | 2 | Q03963 | 54287 | 58 | 62 | 1 | − | 31.10 |
| 9201 | | CANCER | 35 | Rap2 | 2 | 0 | 0 | 21 | 21 | 1 | − | div/0 |
| 6501 | R73920 | CONTROL | 29 | Rho-25 KD | 2 | P03749 | 387 | 21 | 25 | 1 | + | 3.06 |
| 6607 | R41220 | CANCER | 14 | RIP | 2 | Q13546 | 8737 | 74 | 66 | 1 | − | 2.16 |

TABLE 7
Low risk Subjects vs. Stage 0 Cancer Subjects
Level 1 - Changes greater than 2-fold from low quality signals

| | | | | | | Swiss Prot | | Predicted | | | | |
|---|---|---|---|---|---|---|---|---|---|---|---|---|
| SSP | Acc. No. | Type | Lane | Protein Name | Level | ID | Locus | MW | MW | Signal | Change | FoldChange |
| 7406 | 556596 | CANCER | 24 | 14-3-3 | 1 | Q9S928 | 55948 | 30 | 30 | 2 | + | 3.22 |
| 2603 | A27320 | CANCER | 11 | Acetylcholinesterase | 1 | P22303 | 43 | 68 | 60 | 2 | + | 104.25 |
| 8503 | A40720 | CONTROL | 33 | ApoE | 1 | P02649 | 348 | 36 | 36 | 2 | − | div/0 |
| 9503 | A37720 | CANCER | 35 | Arp3 | 1 | 0 | 0 | 50 | 51 | 2 | − | 5.64 |
| 9601 | A37720 | CONTROL | 37 | Arp3 | 1 | 0 | 0 | 50 | 50 | 2 | − | 2.70 |
| 901 | G73320 | CONTROL | 1 | BiP/GRP78 | 1 | P11021 | 3309 | 78 | 74 | 2 | + | 2.24 |
| 9508 | C41720 | CANCER | 35 | Calreticulin | 1 | 0 | 0 | 60 | 60 | 2 | + | 2.26 |
| 7504 | C14520 | CANCER | 22 | Csk | 1 | P32577 | 64019 | 50 | 56 | 2 | − | 50.52 |
| 1701 | C37020 | CANCER | 4 | E-Cadherin | 1 | P12830 | 999 | 120 | 120 | 2 | − | div/0 |
| 2602 | H62120 | CANCER | 10 | hILP/XIAP | 1 | P98170 | 331 | 57 | 60 | 2 | + | 1.99 |
| 1404 | H22020 | CANCER | 5 | Hsp40 | 1 | P25685 | 3301 | 40 | 43 | 2 | + | 2.09 |
| 1501 | M93620 | CANCER | 5 | Mint3/X11g-55KD | 1 | O88888 | 9546 | 61 | 55 | 2 | + | 3.22 |
| | | | | (doublet) | | | | | | | | |
| 4303 | N25720 | CONTROL | 15 | Nm23 | 1 | P15531 | 4831 | 17 | 19 | 2 | + | 2.72 |
| 9702 | N41520 | CANCER | 28 | nNOS/NOS type 1-172KD | 1 | P29476 | 4842 | 155 | 172 | 2 | + | 6.07 |
| 9704 | P49620 | CANCER | 31 | Paxillin-147KD | 1 | P49024 | 5829 | 68 | 147 | 2 | + | 2.51 |
| 9606 | P49620 | CANCER | 31 | Paxillin-75KD | 1 | P49024 | 5829 | 68 | 75 | 2 | − | 3.67 |
| 8502 | P56720 | CONTROL | 34 | PCNA | 1 | P12004 | 5111 | 36 | 28 | 2 | + | 2.24 |
| 8901 | P71720 | CONTROL | 34 | PDI | 1 | P05307 | 64714 | 55 | 55 | 2 | + | 2.00 |
| 4602 | P62220 | CANCER | 12 | PKR/p68 Kinase | 1 | P19525 | 5610 | 68 | 60 | 2 | + | 3.47 |
| 2401 | R56220 | CONTROL | 2 | Rac1 | 1 | P15154 | 207 | 21 | 24 | 2 | + | 2.86 |
| 9302 | | CONTROL | 37 | Rap2 | 1 | 0 | 0 | 21 | 20 | 2 | − | div/0 |
| 2702 | G12920 | CANCER | 9 | Ras-GAP | 1 | P20936 | 5921 | 120 | 128 | 2 | − | div/0 |
| 7902 | R68320 | CONTROL | 32 | Rb | 1 | P13405 | 19645 | 110 | 104 | 2 | + | 3.82 |
| 2901 | G16920 | CONTROL | 5 | Stat1 (N-terminus) | 1 | P42224 | 6772 | 91/84 | 86 | 2 | − | div/0 |
| 603 | T93820 | CANCER | 2 | TLS | 1 | P35637 | 2521 | 65 | 61 | 2 | − | 4.19 |

TABLE 8A
Healthy vs. Cancer (St 0 to St 4)

| | Sensitivity | Specificity | Cutoff | Area Under | Up/Down |
|---|---|---|---|---|---|
| Biomarker | (%) | (%) | (Abs) | ROC | Regulated |
| Ubiquitin | 18.8 | 100 | 1.91 | 0.491 | Upregulated |
| Zn alpha 2 | 34.4 | 86.4 | 3.10 | 0.501 | Upregulated |
| Cytochrome | 28.1 | 95.5 | 2.82 | 0.533 | Down- |
| P450 | | | | | regulated |

TABLE 8B
Healthy vs. Diseased (Benign to St. 4)

| | Sensitivity | Specificity | Cutoff | Area Under | Up/Down |
|---|---|---|---|---|---|
| Biomarker | (%) | (%) | (Abs) | ROC | Regulated |
| Ubiquitin | 37.5 | 100 | 1.91 | 0.644 | Upregulated |
| Zn alpha 2 | 52.1 | 90.9 | 3.07 | 0.668 | Upregulated |
| Cytochrome | 50.0 | 77.3 | 2.19 | 0.587 | Down- |
| P450 | | | | | regulated |

TABLE 8C
Benign vs. Cancer (St. 0 to St. 4)

| | Sensitivity | Specificity | Cutoff | Area Under | Up/Down |
|---|---|---|---|---|---|
| Biomarker | (%) | (%) | (Abs) | ROC | Regulated |
| Ubiquitin | 71.9 | 100 | 1.54 | 0.900 | Upregulated |
| Zn alpha 2 | 96.9 | 100 | 1.81 | 0.969 | Upregulated |
| Cytochrome | 62.5 | 93.7 | 2.31 | 0.773 | Down- |
| P450 | | | | | regulated |

TABLE 8D
Healthy vs. Benign

| | Sensi- | Specif- | | | |
|---|---|---|---|---|---|
| | tivity | icity | Cutoff | Area Under | Up/Down |
| Biomarker | (%) | (%) | (Abs) | ROC | Regulated |
| Ubiquitin | 100 | 77.3 | 1.48 | 0.952 | Upregulated |
| Zn alpha 2 | 100 | 100 | 1.81 | 1.00 | Upregulated |
| Cytochrome | 87.5 | 68.2 | 2.31 | 0.827 | Down- |
| P450 | | | | | regulated |

TABLE 9
Liquid Chromatography Mass Spectrometry/
Mass Spectrometry Experiment Results

| | | Before | | | % Total |
|---|---|---|---|---|---|
| Confidence | Proteins | Grouping | Peptides | Spectra | Spectra |
| >99 | 70 | 966 | 478 | 1099 | 73.6 |
| >95 | 209 | 1674 | 626 | 1329 | 89 |
| >66 | 351 | 1978 | 772 | 1491 | 99.9 |
| As shown: >95 | 209 | 1674 | 626 | 1329 | 89 |

TABLE 10
Comparison of Healthy Subjects, Benign Subjects, and Cancer Groups

| Comparison | Up-Regulated | Down-Regulated | Total Markers |
|---|---|---|---|
| Healthy versus benign | 19 | 10 | 29 |
| Healthy versus stage 0 | 15 | 15 | 30 |
| Healthy versus stage I | 9 | 17 | 26 |
| Benign versus cancers | 9 | 6 | 15 |

TABLE 11
Candidate Up and Down Proteins

| Accession | Protein Name | Ratio | P Value |
|---|---|---|---|
| Q9DCTI | Aldo-keto reductase | 1.3874 | 0.0264 |
| P04083 | Annexin A1 | 3.0606 | 0.0001 |
| P23280 | Carbonic anhydrase VI | 1.5160 | 0.0003 |
| P01040 | Cystatin A | 2.0057 | 0.0014 |
| P01036 | Cystatin SA-III | 1.2030 | 0.0115 |
| Q7NXI3 | Heat shock 70kDa protein | 1.2732 | 0.0039 |
| Q01469 | Epidermal fatty acid-binding protein | 2.0963 | 0.0362 |
| Q6LAF3 | Histone H4 | 2.4094 | 0.0059 |
| P01857 | Ig gamma-1 chain C region | 1.4396 | 0.0034 |
| P13646 | Cytokeratin 13 | 6.5643 | 0.0001 |
| P19013 | Cytokeratin 4 | 6.4958 | 0.0019 |
| P48666 | Cytokeratin 6C | 4.4113 | 0.0001 |
| P01871 | Ig mu chain C region | 1.5134 | 0.0011 |
| Q9HC84 | Mucin 5B | 1.6771 | 0.0001 |
| P05164 | Myeloperoxidase precursor | 2.7188 | 0.0005 |
| P31151 | S100 calcium-binding protein | 2.0519 | 0.0001 |
| P05109 | Calgranulin A | 2.1848 | 0.0001 |
| P06702 | Calgranulin B | 1.8686 | 0.0001 |

TABLE 12
Candidate Up and Down Proteins

| Accession | Protein Name | Ratio | P Value |
|---|---|---|---|
| P10981 | Actin-87E | 0.7657 | 0.0107 |
| Q8N4F0 | Bactericidal/permeability-increasing | 0.7953 | 0.0004 |
| | protein-like 1 | | |
| Q9GQM9 | Cytochrome P450 | 0.7277 | 0.0001 |
| P04264 | Cytokeratin 1 | 0.6106 | 0.0001 |
| P01034 | Cystatin C | 0.7201 | 0.0187 |
| P28325 | Cystatin D precursor | 0.6856 | 0.0010 |
| Q71SP7 | Fatty acid synthase | 0.0311 | 0.0500 |
| P00738 | Haptoglobin | 0.8266 | 0.0023 |
| P22079 | Lactoperoxidase | 0.8247 | 0.0388 |
| Q96DR5 | Lipocalin | 0.6144 | 0.0001 |
| P79180 | Lysozyme C | 0.5309 | 0.0031 |
| P07737 | Profilin-1 | 0.6752 | 0.0135 |
| P02768 | Serum albumin precursor | 0.7336 | 0.0001 |
| P02787 | Transferrin | 0.7192 | 0.0001 |
| P25311 | Zinc-alpha-2-glycoprotein | 0.8454 | 0.0009 |

### REFERENCES

The following references may be helpful for understanding the background of the disclosure.
1. Streckfus C F, Bigler L. The use of soluble, salivary c-erbB-2 for the detection and post-operative follow-up of breast cancer in women: the results of a five year translational study. Adv Dent Res 2005; 18:17-22.
2. Schipper R, Silletti E, Vingerhoeds M H. Saliva as research material: biochemical, physicochemical and practical aspects. Arch Oral Biol 2007; 52:1114-35.
3. Streckfus C F, Bigler L. Saliva as a diagnostic fluid. Oral Dis 2002; 8(2):69-76.
4. Koomen J M, Li D, Xiao L, et al. Direct tandem mass spectrometry reveals limitations in protein profiling experiments for plasma biomarker recovery. J Proteome Res 2005; 4(3):972-81
5. Teisner B, Davey M W, Grudzinskas J G. Interaction between heparin and plasma proteins analyzed by crossed immunoelectrophoresis and affinity chromatography. Clin Chim Acta 1983; 127(3):413-7
6. Srinivas P R, Srivastava S, Hanash S, et al. Proteomics in early detection of cancer. Clin Chem 2001; 47(10):1901-11).
7. Ernst D J, Balance L O. Quality collection: the phlebotomist's role in pre-analytical errors. MLO Med Lab Obs 2006; 83(9):30-8.
8. Schipper R, Loof A, De Groot J, et al. SELDI-TOF-MS of saliva: methodology and pre-treatment effects. J Chromatogr B Analyt Technol Biomed Life Sci 2007; 847: 46-53.
9. Streckfus C F, Bigler L R, Zwick M. The use of surface enhanced laser desorption/ionization time-of-flight mass spectrometry to detect putative breast cancer markers in saliva: a feasibility study. J Oral Pathol Med 2006; 35:292-300.
10. Streckfus C F, Dubinsky W. Proteomic analysis of saliva for cancer diagnosis. Expert Rev Proteomics 2007; 4(3):329-32.
11. Streckfus C F, Mayorga-Wark O, Daniel Arreola D, et al. Breast cancer related proteins are present in saliva and are modulated secondary to ductal carcinoma in situ of the breast. Cancer Invest 2008; 26(2):159-67.
12. Streckfus C F, Bigler L, Dellinger T D, et al. The presence of c-erbB-2, and CA 15-3 in saliva and serum among women with breast carcinoma: a preliminary study. Clin Cancer Res 2000; 6(6):2363-70.
13. Wilmarth P A, Riviere M A, Rustvold D L, et al. Two dimensional liquid chromatography study of the human whole saliva proteome. J Proteome Res 2004; 3:1017-23.
14. Gu S, Liu Z, Pan S, et al. Global investigation of p53-induced apoptosis through quantitative proteomic profiling using comparative amino acid-coded tagging. Mol Cell Proteomics 2004; 10:998-1008.
15. Shevchenko A, Chemushevic I, Shevchenko A, et al. De novo sequencing of peptides recovered from in-gel digested proteins by nanoelectrospray tandem mass spectrometry. Mol Biotechnol 2002; 20:107-18.
16. Koomen J M, Zhao H, Li D, et al. Diagnostic protein discovery using proteolytic peptide targeting and identification. Rapid Commun Mass Spectrom 2004; 18: 2537-48.
17. Ward L D, Reid G E, Moritz R L, et al. Strategies for internal amino acid sequence analysis of proteins separated by polyacrylamide gel electrophoresis. J Chromatogr 1990; 519:199-216.
18. Hu S, Li Y, Wong D T, et al. Large-scale identification of proteins in human salivary proteome by liquid chromatography/mass spectrometry and two-dimensional gel electrophoresis-mass spectrometry. Proteomics 2005; 5:1714-28.
19. Hu S, Loo J A, Wong D T. Human saliva proteome analysis. Ann N Y Acad Sci 2007; 1098:323-9.

While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Accordingly, the scope of protection is not limited by the representative description set out above, but is only limited by the claims which follow, that scope including all equivalents of the subject matter of the claims. The disclosures of all patents, patent applications, and publications cited herein are hereby incorporated herein by reference, to the extent that they provide exemplary, procedural, or other details supplementary to those set forth herein.

What is claimed is:

1. A method of diagnosing a patient's risk of breast cancer, comprising:
(a) measuring in a saliva sample from the patient a concentration of at least a first protein marker, wherein said first protein marker is either cytochrome p450 or ubiquitin, to provide a set of test data comprising a concentration value of each said protein marker in said saliva sample;
(b) comparing said test data to a reference panel comprising a Reference Control Value and a Reference Breast Cancer Value; and
(c) determining a diagnosis of either increased or decreased risk of breast cancer for said patient based on a result of said comparison.

2. The method of claim 1, wherein, in (b), said comparing yields a comparison result in which the concentration value of at least said first protein marker differs from the Reference Control Value of the respective protein marker, wherein said difference is significant at a level in the range of p<0.05 to p<0.0001.

3. The method of claim 1, wherein said Reference Control Value comprises a mean concentration value of each said protein marker in saliva from a group of breast cancer-free individuals, and said Reference Breast Cancer Value comprises a mean concentration value of each said protein in saliva from a group of individuals with breast cancer.

4. The method of claim 3, wherein said Reference Breast Cancer Value comprises a mean concentration value of each said protein in saliva from a group of individuals with ductal carcinoma in situ of breast.

5. The method of claim 1, wherein said at least a first protein marker further comprises at least a second protein marker selected from the group consisting of cytochrome p450, ubiquitin, carbonic anhydrase VI (CAH6), cytokeratin 4 (K2C4), cystatin A (CYTA), epidermal fatty acid-binding protein (FABP4), Ig gamma-1 chain C region (IGHGI), lactoferrin (TRFL), bacterial permeability-increasing protein-1 (BPIL1), haptoglobin (HPT), profilin-1 (PROF1) and zinc-alpha-2-glycoprotein (ZA2G), and
in (c), said determining comprises determining a diagnosis of either increased or decreased risk of breast cancer for said patient based on a result of said comparison.

6. The method of claim 1, wherein
in (a), at least one said protein marker is known to be differentially expressed among benign fibroadenoma, breast cancer and tumor-free breast tissue;
in (b), (2) further comprises:
(2′) a mean concentration value of each said protein marker in saliva from a group of individuals with benign breast tumor (Reference Benign Value), and
in (c), said determining includes differentiating increased risk of breast cancer from increased risk of benign fibroadenoma in the patient.

7. The method of claim 6, wherein said Reference Breast Cancer Value and/or said Reference Benign Value differs from said Reference Control Value, wherein said difference is significant at a level in the range of p<0.05 to p<0.0001.

8. The method of claim 6, wherein said at least a first protein marker comprises at least a second protein marker selected from the group consisting of cytochrome p450, ubiquitin, alpha enolase (ENOA), Ig alpha-2 chain C region (IGHA2), interleukin-1 receptor antagonist protein precursor (IL-1RA), S100 calcium-binding protein A7 (S100A7), short palate, lung and nasal epithelial cancer associated protein 2 (SPLC2), and HER2/neu.
in (c), said determining comprises determining a diagnosis of increased or decreased risk of fibroadenoma in said patient based on a result of said comparison.

9. The method of claim 1, wherein said reference panel is prepared by a process that comprises (b₁) isotopic labeling of salivary proteins and subjecting said labeled proteins to liquid chromatography tandem mass spectrometry to qualitatively and quantitatively characterize salivary proteins of said respective breast cancer and control groups, and (b₂) determining from said characterization a mean concentration value for each said protein marker in said respective cancer and control groups.

10. The method of claim 1, wherein, in (a), said measuring comprises performing an immunoassay.

11. The method of claim 1, wherein said saliva sample is a first saliva sample from said patient, and, in (a), the set of test data is a first set of test data, and the method further comprises:
(d) obtaining a second saliva sample from said patient subsequent to said first sample;
(e) measuring a concentration of at least said first protein marker in the second saliva sample, to provide a second set of test data comprising a second concentration value of each said protein marker in said saliva sample;
(f) comparing the second set of test data to said reference panel; and
(g) determining a second diagnosis of either increased or decreased risk of breast cancer in said patient based on a result of the comparison in (f.

12. The method of claim 1, wherein (f) further comprises comparing said second set of test data to said first set of test data to determine whether a difference in the concentration value of at least said first protein marker exists.

13. The method of claim 12, wherein said first saliva sample is obtained prior to surgical removal of cancerous breast tissue from said patient.

14. The method of claim 12, wherein said patient has received therapeutic treatment for breast cancer prior to obtaining said second saliva sample.

15. The method of claim 14 wherein, in (g), determining said diagnosis comprises an indication whether the therapeutic treatment is effective in said patient.

16. A method of screening a population for increased risk of breast cancer, comprising:
(a) measuring in saliva samples from respective patients a concentration of at least a first protein marker, wherein said first protein marker is either ubiquitin or cytochrome p450, to provide a set of test data comprising a concentration value of each said protein marker in each said saliva sample;
(b) comparing each said set of test data to a reference panel comprising a Reference Control Value and a Reference Breast Cancer Value; and
(c) determining a diagnosis of either increased or decreased risk of breast cancer for each said patient, based on a result of a respective comparison in (b); and
(d) administering a therapeutic treatment to patients with a diagnosis of increased risk of breast cancer, based on a result of said respective comparison.

17. A test kit for identifying a person at increased risk of breast cancer, comprising:
(a) a first set of components for performing a first immunoassay to detect and quantify a first protein marker selected from the group consisting of cytochrome p450 and ubiquitin in saliva; and
(b) at least a second set of components for performing at least a second immunoassay to detect and quantify at least one additional protein marker selected from the group consisting of cytochrome p450, ubiquitin, carbonic anhydrase VI (CAH6), cytokeratin 4 (K2C4), cystatin A (CYTA), epidermal fatty acid-binding protein (FABP4), Ig gamma-1 chain C region (IGHGI), lactoferrin (TRFL), bacterial permeability-increasing protein-1 (BPIL1), haptoglobin (HPT), profilin-1 (PROF1) and zinc-alpha-2-glycoprotein (ZA2G).

18. The test kit of claim 17, wherein said at least a second set of components comprises components for performing a third immunoassay to detect and quantify at least one additional protein marker selected from the group consisting of alpha enolase (ENOA), Ig alpha-2 chain C region (IGHA2), interleukin-1 receptor antagonist protein precursor (IL-1RA), S100 calcium-binding protein A7 (S10A7) and short palate, lung and nasal epithelial cancer associated protein 2 (SPLC2) and HER2/neu.

19. The test kit of claim 17 further comprising (c) at least one control solution containing a protein or peptide to serve as a positive or negative control for each respective immunoassay.
